(19) United States
(12) Patent Application Publication
(10) Pub. No.: US 2009/0005369 A1
(43) Pub. Date: Jan. 1, 2009

(54) Oxazolidinone Compounds and Compositions and Methods Related Thereto

(76) Inventors: Montserrat Cano, Monistrol (ES); Albert Palomer, Barcelona (ES); Antonio Guglietta, Barcelona (ES)

Correspondence Address: BIRCH STEWART KOLASCH & BIRCH, PO BOX 747, FALLS CHURCH, VA, 22040-0747, US

(21) Appl. No.: 11/658,670

(22) PCT Filed: Jul. 26, 2005
(86) PCT No.: PCT/EP05/53627

§ 371 Date: Jul. 24, 2007

(30) Foreign Application Priority Data
Jul. 29, 2004 (EP) 04103657.5

(51) Int. Cl. A61K 31/541; C07D 413/14; A61K 31/496; A61K 31/5377; C07D 417/14

(52) U.S. Cl. 514/227.8; 544/369; 514/254.02; 514/236.8; 544/137; 544/58.2

(57) ABSTRACT

The invention provides new oxazolidinones of formula (I), where R1, R2, R3 and R4 are independently selected from H, F and Cl; A is certain heterocycles optionally substituted; X is selected from O, S, NR8 and CR8R9; R8 and R9 having different meanings; Y is selected from O, S, SO, SO2, NO, NR11 and CR11R12; R11 and R12 having different meanings; and n is selected from 0 and 1. It also provides different processes for the preparation of such compounds. Oxazolidinones compounds of formula (I) are active against Gram-positive and some Gram-negative human and veterinary pathogens with a weak monoamine oxidase (MAO) inhibitory activity. They are useful for the treatment of bacterial infections.

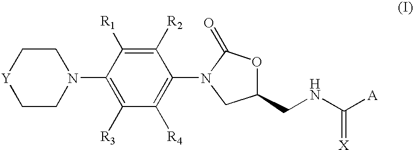

### TECHNICAL FIELD

This invention is directed to oxazolidinone antimicrobial compounds, which are active against Gram-positive and some Gram-negative bacteria with a weak monoamine oxidase (MAO) inhibitory activity.

### BACKGROUND OF THE INVENTION

Oxazolidinones are prominent among the new Gram-positive antimicrobial agents now becoming available. Oxazolidinones bind to the 50S subunit of the prokaryotic ribosome, preventing formation of the initiation complex for protein synthesis. This is a novel mode of action. Other protein synthesis inhibitors either block polypeptide extension or cause misreading of mRNA. Linezolid (N-[[(5S)-3-[3-Fluoro-4-(4-morpholinyl)phenyl]-2-oxo-5-oxazolidinyl]methyl]acetamide) is the first antimicrobial oxazolidinone to be approved for clinical use in the United States and elsewhere.

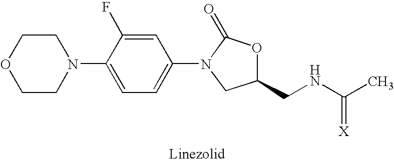

Linezolid minimal inhibitory concentrations (MICs) vary slightly with the test mode, laboratory, and significance attributed to thin hazes of bacterial survival, but all workers find the susceptibility distributions are narrow and unimodal with MIC values between 0.5 and 4 μg/mL for streptococci, enterococci and staphylococci. Full activity is retained against Gram-positive cocci resistant to other antibiotics, including methicillin-resistant staphylococci and vancomycin-resistant enterococci. MICs are 2-8 μg/mL for Moxarella, Pasteurella and Bacteroides spp. but other Gram-negative bacteria are resistant as a result of endogenous efflux activity as well as the intake presented by Gram-negative bacteria outer membrane cell.

Linezolid is indicated for the treatment of adult patients with the following infections:

Vancomydn-resistant Enterococcus faecium infections, including concurrent bacteremia;

nosocomial pneumonia;

complicated skin and skin structure infections;

community-acquired pneumonia, including concurrent bacteremia;

diabetic foot infections; and

uncomplicated skin and skin structure infections.

Oxazolidinones were originally developed as MAOI for treatment of depression and Parkinson's disease. MAO is one of the primary enzymes responsible for the catabolism of catecholamines. In humans, MAO occurs in two isoforms, MAO-A and MAO-B. MAO-A preferentially deaminates serotonin (5-Hr) and norepinephrine; MAO-B preferentially deaminates phenylethylamine, benzylamine, and, in man, dopamine. Normally MAO-A inhibitors, such as moclobemide or tranylcypromine, have been used as antidepressant agents while MAO-B inhibitors, such as selegiline, have been used preferably in the therapy of Parkinson's disease. U.S. Pat. No. 3,655,687 discloses 5-hydroxymethyl-3-substituted-2-oxazolidinone derivatives with significant antidepressant activity. A compound disclosed in this patent, toloxatone, is of particular reference.

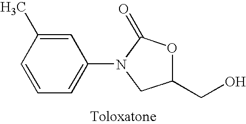

Toloxatone is a selective, reversible inhibitor of MAO-A and has been introduced in clinical practice. Because of this reason, particular attention has been paid to the question of whether evidence of adverse interaction with drugs known to be metabolized by monoamine oxidase would occur in patients treated with linezolid. An enhanced pressor response has been seen in patients taking certain adrenergic agents, including phenylpropanolamine and pseudoephedrine, and it is specifically noted that the doses of these drugs should be reduced in patients receiving linezolid. Animal studies suggest that linezolid moderately potentiates the pressor effects of the endogenous and dietary amine, tyramine, and other sympathomimetic amines. The package insert for linezolid warns against combining it with tyramine-rich foods and about being aware of a potential interaction with adrenergic and serotonergic agents. Accordingly, there is a need of new oxazolidinone antimicrobial compounds with minimum MAO inhibitory activity to eliminate the related side effects from potential drug-drug interactions.

The preparation of linezolid is disclosed in PCT application WO 9507271.

PCT application WO 03084534 discloses a method for treating a diabetic foot infection with oxazolidinones, specially with 3-{4-[1-(2,3-dihydroxy-propionyl)-1,2,3,6-tetrahydro-pyridin-4-yl]-3,5-difluoro-phenyl}-5-(isoxazol-3-yloxymethyl)-oxazolidin-2-one; 2,2-difluoro-N-({(5S)-3-[3-fluoro-4-(4-glycoloylpiperazin-1-yl)phenyl]-2-oxo-1,3-oxazolidin-5-yl}methyl)ethanethioamide; and linezolid.

PCT application WO 03063862 discloses a method of treating a patient in need of oxazolidinone by administering an effective amount of oxazolidinone and an effective amount of at least one vitamin selected from the group consisting of vitamin B2, vitamin B6, vitamin B12 and folic acid.

Patent applications DE 10105989 and US 2003/0153610 disclose the preparation of the N-((2-oxo-3-phenyl-1,3-oxazolidin-5-yl)-methyl)-heterocyclic amides and their use for inhibiting blood coagulation in vitro, especially in preserved blood or biological samples containing factor Xa. Heterocyclic amides disclosed in US 2003/0153610 are limited to thienyl amides, while DE 10105989 focuses on N-[[3-[(4-substituted)-phenyl]-2-oxo-5-oxazolidinyl]methyl]-amides with substituents containing either the oxo- or N-oxide moiety. Moreover, these documents describe neither antibacterial nor MAOi activity.

### SUMMARY OF THE INVENTION

Inventors have surprisingly found that furyl amide compounds of the class disclosed in the present application are particularly active antimicrobial agents showing a weak MAO inhibitory activity. The structures disclosed in the present application clearly differentiate from the compounds in DE 10105989 and US 2003/0153610.

On the whole the present invention provides evidence that new furyl amides of N-[[(3-[4-substituted-phenyl]-2-oxo-5-oxazolidinyl]methyl]-amines are specifically active against Gram-positive human and veterinary pathogens with a weak monoamine oxidase inhibitory activity.

Thus, an aspect of the present invention is the provision of new oxazolidinones specifically active against Gram-positive and some Gram-negative human and veterinary pathogens with a weak monoamine oxidase (MAO) inhibitory activity.

The compounds of the present invention are those of general formula (I), or a pharmaceutically acceptable salt thereof;

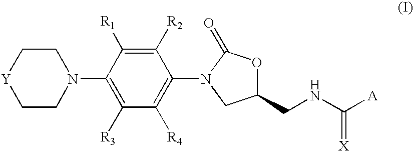

wherein:

—R₁, —R₂, —R₃ and —R₄ are radicals independently selected from hydrogen, F and Cl;

-A is a radical selected from the group consisting of

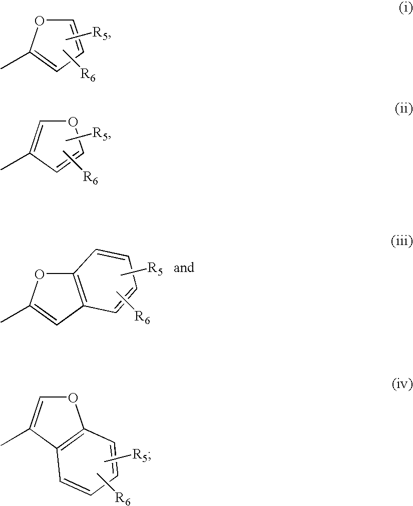

—R₅ and —R₆ are radicals independently selected from the group consisting of hydrogen, F, Cl, Br, —NO₂, —CN, —COR₇, —CSR₇, —SO₂R₇, —OCOR₇, alkyl(C₁-C₆), haloalkyl(C₁-C₆), cycloalkyl(C₃-C₆), alkenyl(C₂-C₆), alkynyl(C₂-C₆), alkoxyl(C₁-C₆), alkoxyalkyl(C₁-C₆), —NH-alkyl(C₁-C₆), —N-dialkyl(C₁-C₆), phenyl optionally substituted and heteroaryl optionally substituted; or —R₅ and —R₆ taken together form an optionally substituted benzo-fused ring;

—R₇ is a radical selected from the group consisting of hydrogen, alkyl(C₁-C₆), cycloalkyl(C₃-C₆), alkenyl(C₂-C₆), alkynyl(C₂-C₆), alkoxyl(C₁-C₆), alkoxyalkyl(C₁-C₆), hydroxyalkyl(C₁-C₆), —NH-alkyl(C₁-C₆), —N-dialkyl(C₁-C₆), phenyl optionally substituted and heteroaryl optionally substituted;

X is selected from O, S, NR₈ and CR₈R₉;

—R₈ and —R₉ are radicals independently selected from the group consisting of hydrogen, —CN, —COR₁₀, —SO₂R₁₀, alkyl(C₁-C₆), haloalkyl(C₁-C₆), cycloalkyl(C₃-C₆), alkenyl(C₂-C₆), alkynyl(C₂-C₆), alkoxyl(C₁-C₆), alkoxyalkyl(C₁-C₆), —NH-alkyl(C₁-C₆), —N-dialkyl(C₁-C₆), phenyl optionally substituted and heteroaryl optionally substituted;

—R₁₀ is a radical selected from the group consisting of hydrogen, alkyl(C₁-C₆), -haloalkyl(C₁-C₆), cycloalkyl(C₃-C₆), alkenyl(C₂-C₆), alkynyl(C₂-C₆), alkoxyalkyl(C₁-C₆), phenyl optionally substituted and heteroaryl optionally substituted;

—Y— is a biradical selected from O, S, SO, SO₂, NO, NR₁₁, and CR₁₁R₁₂;

—R₁₁ and —R₁₂ are a radical independently selected from the group consisting of hydrogen, —(CHR₁₃)ₙR₁₄, —CN, —COR₁₃, —CSR₁₃, —COOR₁₃, —CSOR₁₃, —CONR₁₃R₁₄, —CSNR₁₃R₁₄, —CON(R₁₅)N(R₁₄)R₁₃, —SO₂R₁₃, —SO₂OR₁₃, —SO₂NR₁₃R₁₄, alkyl(C₁-C₆), haloalkyl(C₁-C₆), cycloalkyl(C₃-C₆), alkenyl(C₂-C₆), alkynyl(C₂-C₆), alkoxyalkyl(C₁-C₆), phenyl optionally substituted and heteroaryl optionally substituted;

n is selected from 0 and 1;

—R₁₃ and —R₁₄ are a radical independently selected from the group consisting of hydrogen, —COR₁₅, —CSR₁₅, —SO₂R₁₅, alkyl(C₁-C₆), cycloalkyl(C₃-C₆), alkenyl(C₂-C₆), alkynyl(C₂-C₆), alkoxyl(C₁-C₆), alkoxyalkyl(C₁-C₆), hydroxyalkyl(C₁-C₆), dihydroxyalkyl(C₁-C₆), phenyl optionally substituted,

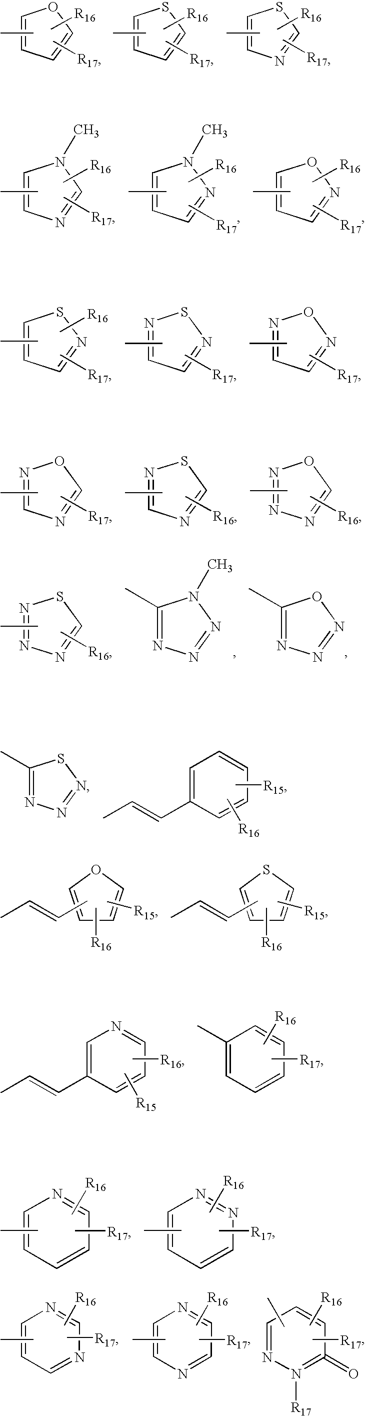

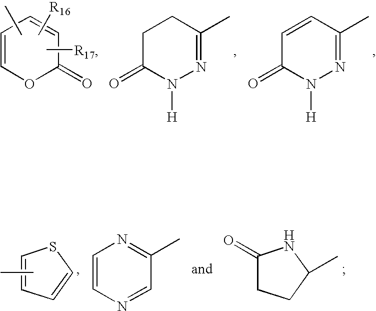

—R₁₅ is a radical selected from the group consisting of hydrogen, alkyl(C₁-C₆), cycloalkyl(C₃-C₆), alkenyl(C₂-C₆), alkynyl(C₂-C₆), alkoxyl(C₁-C₆), alkoxyalkyl(C₁-C₆), hydroxyalkyl(C₁-C₆), phenyl optionally substituted and heteroaryl optionally substituted;

—R₁₆ and —R₁₇ are radicals independently selected from the group consisting of F, Cl, Br, —NO₂, —CN, —COR₁₈, —CONR₁₈R₁₉, —SO₂R₁₈, —SO₂NR₁₈R₁₉, alkyl(C₁-C₆), haloalkyl(C₁-C₆), cycloalkyl(C₃-C₆), alkenyl(C₂-C₆), alkynyl(C₂-C₆), alkoxy(C₁-C₆), alkoxyalkyl(C₁-C₆), phenyl optionally substituted and heteroaryl optionally substituted; and

—R₁₈ and —R₁₉ are radicals independently selected from the group consisting of hydrogen, alkyl(C₁-C₆), haloalkyl(C₁-C₆), cycloalkyl(C₃-C₆), alkenyl(C₂-C₆), alkynyl(C₂-C₆), alkoxyl(C₁-C₆), alkoxyalkyl(C₁-C₆), phenyl optionally substituted and heteroaryl optionally substituted.

Another aspect of the invention relates to methods for preparing the compounds of formula (I) which comprises:

(a) Preparation of amide compounds (I, X═O) by acylating the amino methyl intermediates of general formula (II):

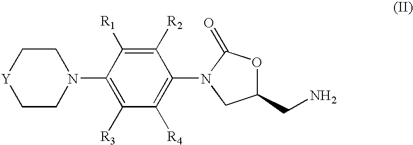

wherein —R₁, —R₂, —R₃ and —R₄ and —Y— are as defined in the general formula (I), with an activated form of the corresponding acid (III):

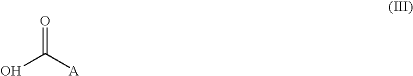

wherein -A is as defined in the general formula (I);

(b) Preparation of thioamide compounds (I, X═S) from the corresponding amides (I, X═O) by reacting with a thionation reagent or by condensing the corresponding methyl amine (II) with an alkyldithioamide (IIIi):

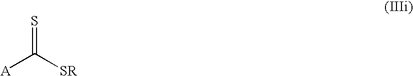

wherein -A is as defined in the general formula (I) and —R is an alkyl(C₁-C₆);

(c) Preparation of sulfoxide (I, Y═SO) or sulfone compounds (I, Y═SO₂) from the corresponding sulfide compounds (I, Y═S) by reacting with an oxidation reagent, depending the obtained compound on the nature of said reagent;

(d) Preparation of cyanoamidine compounds (I, X═N—CN) by reacting the amino methyl intermediates of general formula (II) with an appropriate alkyl cyanoimidate of general formula (V):

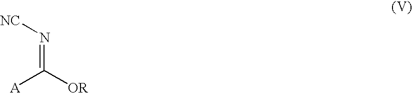

wherein -A is as defined in the general formula (I) and —R is an alkyl(C₁-C₆).

(e) Preparation of amide compounds (I, X═O; Y═NH) by acylating the amino methyl intermediates of general formula (IIa):

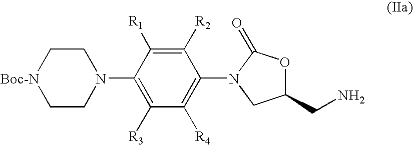

wherein —R₁, —R₂, —R₃ and —R₄ are as defined above and Boc is a t-butoxycarbonyl N-protecting group, with the corresponding acid of formula (III) in the presence of 3-dimethylaminopropyl-3-ethyl-carbodiimide hydrochloride and 4-(dimethylamino)pyridine through the intermediate compound of formula (Ia):

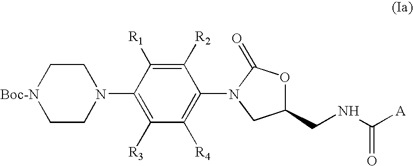

wherein -A, Boc, —R₁, —R₂, —R₃, and —R₄ are as defined above, and subsequent splitting off the Boc N-protecting group with trifluoroacetic acid.

(f) Preparation of amide compounds (I, X═O; Y═NCOR₁₃) by reacting a compound of general formula (I), when X is O and —Y— is NH, with an activated form of the corresponding acid of formula (VI):

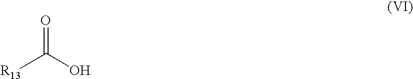

wherein —R₁₃ is as defined above.

In the present invention activated forms of carboxylic acids stand for acid halides, imidazolides, p-nitrophenyl esters and 2,4,5-trichlorophenyl esters thereof. The activated forms of carboxylic acids are prepared in situ in the presence of a reagent selected from triphenylphosphine, bromotrichloromethane, dicyclohexylcarbodiimide, 2-chloropyridinium cation, 3-chloroisoxazolium cation, diphenylphosphoryl azide, N-hydroxybenzotriazole (HOBt), 2-(1H-benzotriazole-1-yl)-1,1,3,3-tetramethyluronium hexafluorophosphate (HBTU), 1-(mesitylene-2-sulfonyl)-3-nitro-1H-1,2,4-triazole (MSNT), benzotriazole-1-yl-oxy-trispyrrolidino-phosphonium hexafluorophosphate (PyBOP), 1-ethyl-3-(3′-dimethylaminopropyl)carbodiimide HCl (WSC.HCl) and 2-(1H-benzotriazole-1-yl)-1,1,3,3-tetramethyluronium tetrafluoroborate (TBTU), and the like.

Another aspect of the invention relates to a pharmaceutical composition comprising a therapeutically effective amount of the compound of general formula (I) as defined above, together with the appropriate amounts of pharmaceutical excipients or carriers.

Another aspect of the invention relates to the use of a compound of formula (I) for the preparation of a pharmaceutical composition to treat bacterial infections in a human or animal.

The pharmaceutical composition of the present invention can be administered by oral, parenteral, inhalatory, rectal, transdermal or topical administration, being the compound of general formula (I) administered in an amount of 0.1 to 100 mg/kg of body weight/day, preferably 1 to 50 mg/kg of body weight/day.

Another aspect of the invention relates to a method of treatment of a mammal, including a human, suffering from a bacterial infection. This method comprises the administration of a therapeutically effective amount of a compound of formula (I) as defined above, together with pharmaceutically acceptable diluents or carriers, to said patients.

### DETAILED DESCRIPTION OF THE INVENTION

The present invention relates to novel oxazolidinone compounds of formula (I) or a pharmaceutically acceptable salt thereof;

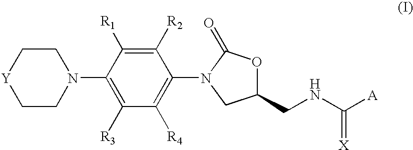

wherein:

—R₁, —R₂, —R₃ and —R₄ are radicals independently selected from hydrogen, F and Cl;

-A is a radical selected from the group consisting of

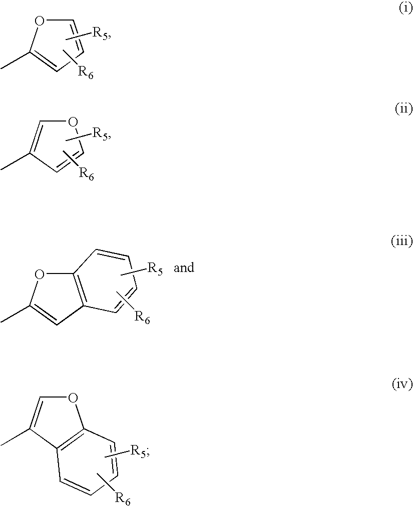

—R₅ and —R₆ are a radical independently selected from the group consisting of hydrogen, F, Cl, Br, —NO₂, —CN, —COR₇, —CSR₇, —SO₂R₇, —OCOR₇, alkyl(C₁-C₆), haloalkyl(C₁-C₆), cycloalkyl(C₃-C₆), alkenyl(C₂-C₆), alkynyl(C₂-C₆), alkoxyl(C₁-C₆), alkoxyalkyl(C₁-C₆), —NH-alkyl(C₁-C₆), —N-dialkyl(C₁-C₆), phenyl and heteroaryl; or R₅ and R₆ taken together form taken together form an optionally substituted benzo-fused ring optionally substituted;

—R₇ is a radical selected from the group consisting of hydrogen, alkyl(C₁-C₆), cycloalkyl(C₃-C₆), alkenyl(C₂-C₆), alkynyl(C₂-C₆), alkoxyl(C₁-C₆), alkoxyalkyl(C₁-C₆), hydroxyalkyl(C₁-C₆), —NH-alkyl(C₁-C₆), —N-dialkyl(C₁-C₆), phenyl and heteroaryl;

X is selected from O, S, NR₈ and CR₈R₉;

—R₈ and —R₉ are radicals independently selected from the group consisting of hydrogen, —CN, —COR₁₀, —SO₂R₁₀, alkyl(C₁-C₆), haloalkyl(C₁-C₆), cycloalkyl(C₃-C₆), alkenyl(C₂-C₆), alkynyl(C₂-C₆), alkoxyl(C₁-C₆), alkoxyalkyl(C₁-C₆), —NH-alkyl(C₁-C₆), —N-dialkyl(C₁-C₆), phenyl and heteroaryl;

—R₁₀ is a radical selected from the group consisting of hydrogen, alkyl(C₁-C₆), haloalkyl(C₁-C₆), cycloalkyl(C₃-C₆), alkenyl(C₂-C₆), alkynyl(C₂-C₆), alkoxyalkyl(C₁-C₆), phenyl and heteroaryl;

—Y— is a biradical selected from O, S, SO, SO₂, NO, NR₁₁ and CR₁₁R₁₂;

—R₁₁ and —R₁₂ are a radical independently selected from the group consisting of hydrogen, —(CHR₁₃)ₙR₁₄, —CN, —COR₁₃, —CSR₁₃, —COOR₁₃, —CSOR₁₃, —CONR₁₃R₁₄, —CSNR₁₃R₁₄, —CON(R₁₅)N(R₁₄)R₁₃, —SO₂R₁₃, —SO₂OR₁₃, —SO₂NR₁₃R₁₄, alkyl(C₁-C₆), haloalkyl(C₁-C₆), cycloalkyl(C₃-C₆), alkenyl(C₂-C₆), alkynyl(C₂-C₆), alkoxyalkyl(C₁-C₆), phenyl and heteroaryl;

n is selected from 0 and 1;

—R₁₃ and —R₁₄ are a radical independently selected from the group consisting of hydrogen, —COR₁₅, —CSR₁₅, —SO₂R₁₅, alkyl(C₁-C₆), cycloalkyl(C₃-C₆), alkenyl(C₂-C₆), alkynyl(C₂-C₆), alkoxyl(C₁-C₆), alkoxyalkyl(C₁-C₆), hydroxyalkyl(C₁-C₆), phenyl,

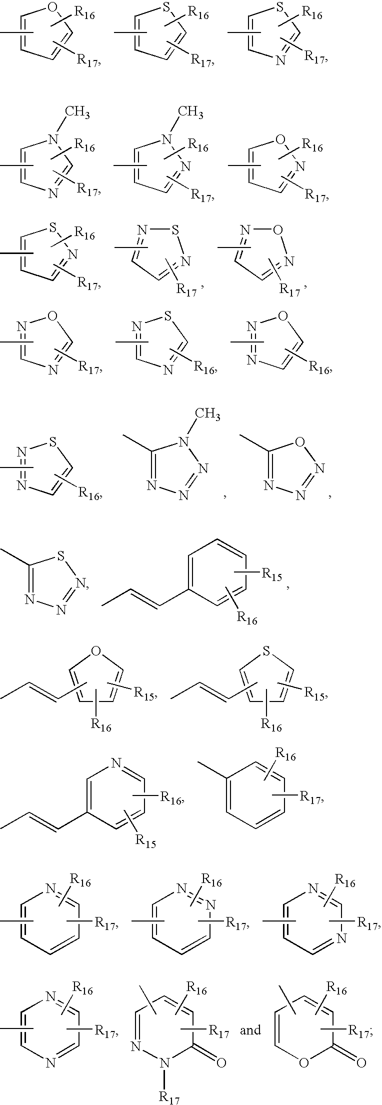

—R₁₅ is a radical selected from the group consisting of hydrogen, alkyl(C₁-C₆), cycloalkyl(C₃-C₆), alkenyl(C₂-C₆), alkynyl(C₂-C₆), alkoxyl(C₁-C₆), alkoxyalkyl(C₁-C₆), hydroxyalkyl(C₁-C₆), phenyl and heteroaryl;

—R₁₆ and —R₁₇ are radicals independently selected from the group consisting of F, Cl, Br, —NO₂, —CN, —COR₁₈, —CONR₁₈R₁₉, —SO₂R₁₈, —SO₂NR₁₈R₁₉, alkyl(C₁-C₆), haloalkyl(C₁-C₆), cycloalkyl(C₃-C₆), alkenyl(C₂-C₆), alkynyl(C₂-C₆), alkoxyl(C₁-C₆), alkoxyalkyl(C₁-C₆), phenyl and heteroaryl;

—R₁₈ and —R₁₉ are radicals independently selected from the group consisting of hydrogen, alkyl(C₁-C₆), haloalkyl(C₁-C₆), cycloalkyl(C₃-C₆), alkenyl(C₂-C₆), alkynyl(C₂-C₆), alkoxyl(C₁-C₆), alkoxyalkyl(C₁-C₆), phenyl and heteroaryl.

Preferably, the present invention relates to new oxazolidinones of formula (I) wherein —R₂, —R₃ and —R₄ are hydrogen and —R₁ is F; X is selected from O, S and N—CN; -A is selected from the group consisting of:

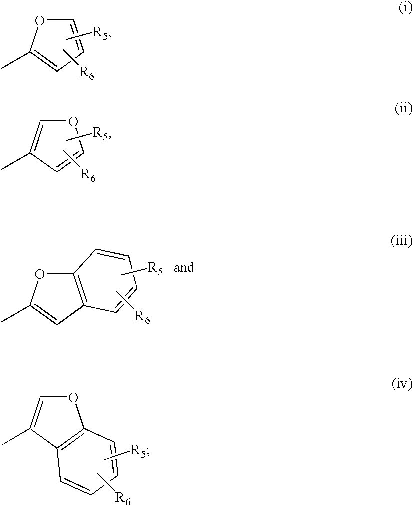

—R₅ and —R₆ are hydrogen, F, Cl, Br and NO₂; —Y— is O, S, SO, SO₂ and NR₁₁; —R₁₁ is hydrogen, methyl, —CN, —COCH₃, —COOCH₃, —CONHCH₃, —SO₂CH₃, —SO₂NHCH₃, —CSCH₃, —CO—(CH₂)₂—OH, —CO—CH₂—OCH₃, —CO—CH═CH₂, —CO—CH₂—OH and —CS—CH₂—OH.

The term “pharmaceutically acceptable salts” used herein encompasses any salt formed from organic and inorganic acids, such as hydrobromic, hydrochloric, phosphoric, nitric, sulfuric, acetic, adipic, aspartic, benzenesulfonic, benzoic, citric, ethanesulfonic, formic, fumaric, glutamic, lactic, maleic, malic, malonic, mandelic, methanesulfonic, 1,5-naphthalendisulfonic, oxalic, pivalic, propionic, p-toluenesulfonic, succinic, tartaric and the like.

The compounds are useful antimicrobial agents, effective against a number of human and veterinary microorganisms. The compounds of the present invention exhibit a weak MAO inhibitory activity, which indicates that these compounds possess the ability to minimize or eliminate potential drug-drug interactions since strong inhibition of monoamine oxidase can result in altered clearance rates for other compounds normally metabolized by monoamine oxidase, including several pharmaceuticals. In addition, it is of particular relevance to avoid increased levels of neurotransmitter amines, such as dopamine, serotonin and noradrenaline.

The preferred compounds of the present invention are:
N-[[(5S)-3-[3-fluoro-4-morpholin-4-yl-phenyl]-2-oxo-5-oxazolidinyl]methyl]furan-2-yl-thioamide;
N-[[(5S)-3-[3-fluoro-4-morpholin-4-yl-phenyl]-2-oxo-5-oxazolidinyl]methyl]furan-3-yl-thioamide
N-[[(5S)-3-[3-fluoro-4-(4′-thioacetyl-4-piperazinyl)-phenyl]-2-oxo-5-oxazolidinyl]methyl]furan-3-yl-thioamide;
N-[[(5S)-3-[3-fluoro-4-thiomorpholin-4-yl-phenyl]-2-oxo-5-oxazolidinyl]methyl]furan-2-yl-thioamide;
N-[[(5S)-3-[3-fluoro-4-thiomorpholin-4-yl-phenyl]-2-oxo-5-oxazolidinyl]methyl]furan-3-yl-thioamide;
N-[[(5S)-3-[3-fluoro-4-morpholin-4-yl-phenyl]-2-oxo-5-oxazolidinyl]methyl]benzofuran-2-yl-amide;
N-[[(5S)-3-[3-fluoro-4-morpholin-4-yl-phenyl]-2-oxo-5-oxazolidinyl]methyl]benzofuran-3-yl-amide;
N-[[(5S)-3-[3-fluoro-4-morpholin-4-yl-phenyl]-2-oxo-5-oxazolidinyl]methyl]5-nitro-benzofuran-2-yl-amide;
N-[[(5S)-3-[3-fluoro-4-(4′-methoxyacetyl-4-piperazinyl)-phenyl]-2-oxo-5-oxazolidinyl]methyl]furan-3-yl-amide;
N-[[(5S)-3-[3-fluoro-4-(4′-acryloyl-4-piperazinyl)-phenyl]-2-oxo-5-oxazolidinyl]methyl]furan-3-yl-amide; and
N-[[(5S)-3-[3-fluoro-4-(4′-hydroxyacetyl-4-piperazinyl)-phenyl]-2-oxo-5-oxazolidinyl]methyl]furan-3-yl-amide.

The compounds of general formula (I) may be prepared by several different methods, depending on the nature of the functional groups:

(a) Preparation of Amide Compounds (I, X═O):

Formally, amides are prepared by condensation of an activated form of the acid (III) with the corresponding amino methyl derivative (II). The acid can be previously converted into a reactive acylating reagent through isolation or preparation in situ. Acid halides, imidazolides and p-nitrophenyl esters or 2,4,5-trichlorophenyl esters are the more common isolable acylating substances prepared directly from carboxylic acid. There are activation procedures, which generate acyl halides in situ in the presence of the nucleophile, such as, refluxing the carboxylic acid, triphenylphosphine, bromotrichloromethane and the amine. The other coupling reagents convert the carboxylic acid into an activated intermediate for reaction with the nucleophilic amine. A wide variety of such reagents can be used, some of them are the following: dicyclohexylcarbodiimide, 2-chloropyridinium cation, 3-chloroisoxazolium cation, diphenylphosphoryl azide, N-hydroxybenzotriazole (HOBt), 2-(1H-benzotriazol-1-yl)-1,1,3,3-tetramethyluronium hexafluorophosphate (HBTU), 1-(mesitylene-2-sulfonyl)-3-nitro-1H-1,2,4-triazole (MSNT), benzotriazol-1-yl-oxy-trispyrrolidino-phosphonium hexafluorophosphate (PyBOP), 1-ethyl-3-(3′-dimethylaminopropyl)carbodiimide HCl (WSC.HCl), 2-(1H-benzotriazol-1-yl)-1,1,3,3-tetramethyluronium tetrafluoroborate (TBTU), and the like. An illustrative convenient procedure for the preparation of the amides of the present invention is shown in the following reaction scheme, wherein 1-ethyl-3-(3′-dimethylaminopropyl)carbodiimide HCl is the activating agent for the acid (III) and 4-(dimethylamino)pyridine acts as a base:

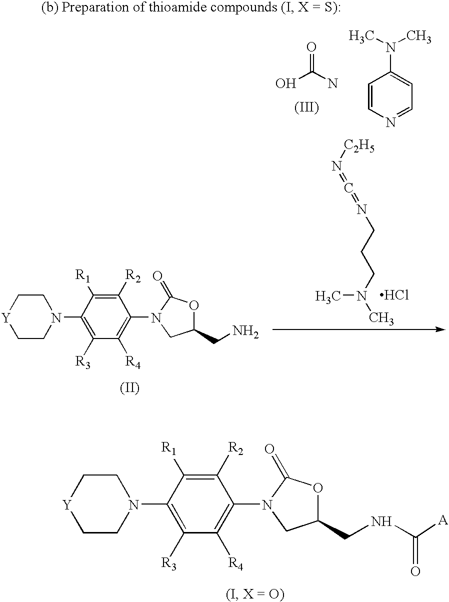

The preparation of the thioamide compounds from the corresponding amide derivatives (I) can be performed by several thionation reagents, such as Lawesson's reagent (IVi) as shown below.

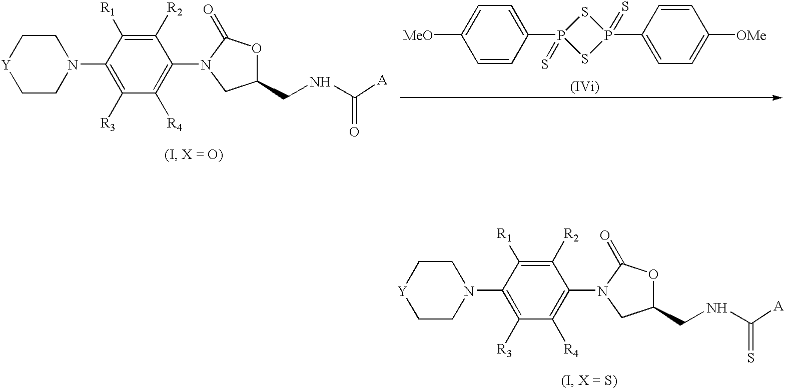

Other examples of thionation reagents are Davy's (IVii), Yokoyama's (CAPLUS 1985:166850), Belleau's (IViii), P₄S₁₀ (IViv), Na₂P₄S₁₁, (IVv), Na₂P₄S₁₀O (IVvi) and the like.

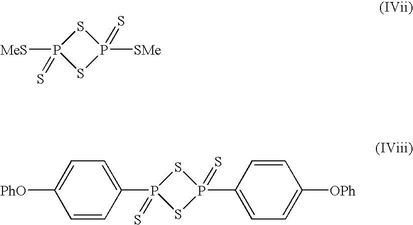

Otherwise, the thioamide compound can be obtained by condensation of the corresponding amino methyl derivative (II) with an alkyldithioamide (IIIi)

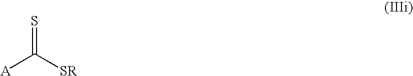

derived from the acid (III) and wherein A is as defined in the general formula (I) and R is an alkyl(C₁-C₆).

(c) Preparation of Sulfoxide Compounds (I, Y═SO):

The preparation of the sulfoxide compounds from the corresponding sulphide (I, Y═S) can be performed by several oxidizing reagents: sodium metaperiodate, the most widely used, as shown below, hypervalent iodine reagents, chromic acid in acetic acid or pyridine, lead tetraacetate, manganese dioxide, thallium (III) nitrate, ozone and the like.

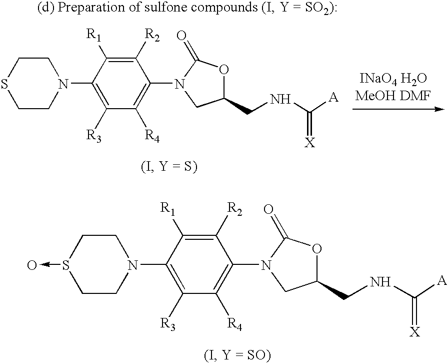

The preparation of the sulfone compounds from the corresponding sulphide (I, Y═S) can be performed by several oxidizing reagents, such as, excess of hydrogen peroxide in acetic acid, the most widely used, as shown below, catalytic osmium tetroxide in the presence of N-methylmorpholine N-oxide, and the like.

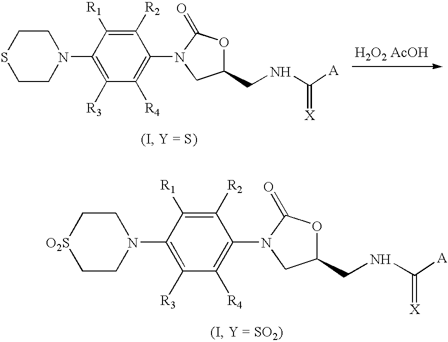

(e) Preparation of Cyanoamidine Compounds (I, X═N—CN):

The cyanoamidine compounds are synthesized by reacting the corresponding amino methyl derivative (II) with the appropriate alkyl N-cyanoimidate (V) wherein A is as defined in the general formula (I) and R is an alkyl(C₁-C₆).

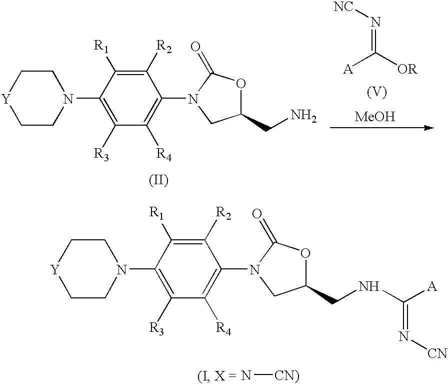

In turn, alkyl N-cyanoimidates can be obtained from the corresponding nitrite by formation of the imidate followed by cyanoamide displacement.

(f) Preparation of Amide Compounds (I, X═O; Y═NH):

Such amide compounds are prepared by acylating an amino methyl intermediate of general formula (IIa)

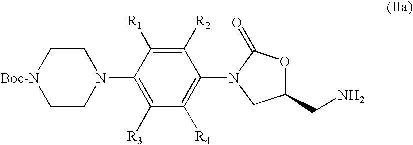

wherein R₁, R₂, R₃ and R₄ are as defined above and Boc is a t-butoxycarbonyl N-protecting group, with the corresponding acid of formula (III) in the presence of 3-dimethylaminopropyl-3-ethyl-carbodiimide hydrochloride and 4-(dimethylamino)pyridine through the intermediate compound of formula (Ia)

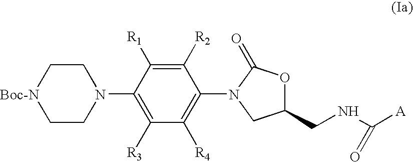

wherein A, Boc, R₁, R₂, R₃, and R₄ are as defined above, and subsequent splitting off the Boc N-protecting group with trifluoroacetic acid.

(g) Preparation of Amide Compounds (I, X═O; Y═NCOR₁₃):

Such amide compounds are prepared by reacting a compound of general formula (I), when X is O and Y is NH, with an activated form of the corresponding acid of formula (VI)

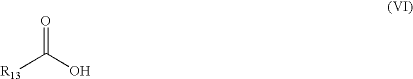

wherein R₁₃ is as defined above.

In the present invention activated forms of carboxylic acids stand for acid halides, imidazolides, p-nitrophenyl esters and 2,4,5-trichlorophenyl esters thereof. The activated forms of carboxylic acids are prepared in situ in the presence of a reagent selected from triphenylphosphine, bromotrichtoromethane, dicyclohexylcarbodiimide, 2-chloropyridinium cation, 3-chloroisoxazolium cation, diphenylphosphoryl azide, N-hydroxybenzotriazole (HOBt), 2-(1H-benzotriazole-1-yl)-1,1,3,3-tetramethyluronium hexafluorophosphate (HBTU), 1-(mesitylene-2-sulfonyl)-3-nitro-1H-1,2,4-triazole (MSNT), benzotriazole-1-yl-oxy-trispyrrolidino-phosphonium hexafluorophosphate (PyBOP), 1-ethyl-3-(3′-dimethylaminopropyl)carbodiimide HCl (WSC.HCl) and 2-(1H-benzotriazole-1-yl)-1,1,3,3-tetramethyluronium tetrafluoroborate (TBTU), and the like.

Certain amino methyl intermediates of general formula (II) are known in the art and may be prepared according to methods disclosed in the literature. Thus, PCT application WO 9507271 discloses the preparation of N-[[(5S)-3-[3-fluoro-4-(4-morpholinyl)-phenyl]-2-oxo-5-oxazolidinyl]methyl]amine (II, R₁═F, R₂═R₃═R₄═H, Y═O), PCT application WO 9854161 discloses the preparation of N-[[(5S)-3-[3-fluoro-4-(4-thiomorpholinyl)-phenyl]-2-oxo-5-oxazolidinyl]methyl]amine (II, R₁═F, R₂═R₃═R₄═H, Y═S) and PCT application WO 0032599 discloses the preparation of N-[[(5S)-3-[3-fluoro-4-(4′-acetyl-4-piperazinyl)-phenyl]-2-oxo-5-oxazolidinyl]methyl]amine (II, R₁═F, R₂═R₃═R₄═H, Y═CH3-CON). PCT application WO 04/018439 discloses the preparation of (S)—N-[3-[3-fluoro-4-[N-t-butoxycarbonylpiperazin-1-yl]phenyl]-2-oxooxazolidin-5-ylmethyl]azide and (S)-[3-[3-fluoro-4-[N-t-butoxycarbonylpiperazin-1-yl]phenyl]-2-oxooxazolidin-5-ylmethyl]alcohol.

The compounds of the present invention can be normally formulated in accordance with standard pharmaceutical practice as a pharmaceutical composition.

The pharmaceutical compositions of this invention may be administered in standard manner for the disease condition that it is desired to treat, for example by oral, parenteral, inhalatory, rectal, transdermal or topical administration. For these purposes the compounds of this invention may be formulated by means known in the art in the form of, for example, tablets, capsules, syrups, aqueous or oily solutions or suspensions, emulsions, dispersible powders, inhalatory solutions, suppositories, ointments, creams, drops and sterile aqueous or oily solutions or suspensions for injection and the like. The pharmaceutical compositions may contain flavoring agents, sweeteners, etc. in suitable solid or liquid carriers or diluents, or in a suitable sterile media to form suspensions or solutions suitable for intravenous, subcutaneous or intramuscular injection. Such compositions typically contain from 1 to 40%, preferably 1 to 10% by weight of active compound, the remainder of the composition being pharmaceutically acceptable carriers, diluents, solvents and the like.

The compounds of formula (I) are administered in an amount of 0.1 to 100 mg/kg of body weight/day, preferably 1 to 50 mg/kg of body weight/day.

The compounds of the present invention are useful in the treatment of conditions such as nosocomial pneumoniae, community acquired pneumoniae, including concurrent bacteremia, vancomydn resistance enterocci (VRE) caused by methicillin resistance staphylococcus aureus (MRSA), including concurrent bacteremia, penicillin resistance streptococcus pneumoniae, diabetic foot infections and skin and skin structure infections.

The compounds of the present invention are effective against a number of human or animal pathogens, clinical isolates, including vancomycin-resistant organisms and methicillin-resistant organisms.

The following non-limiting examples illustrate the scope of the present invention.

### EXAMPLE 1

### N-[[(5S)-3-[3-fluoro-4-(4-morpholinyl)-phenyl]-2-oxo-5-oxazolidinyl]methyl]furan-2-yl-amide

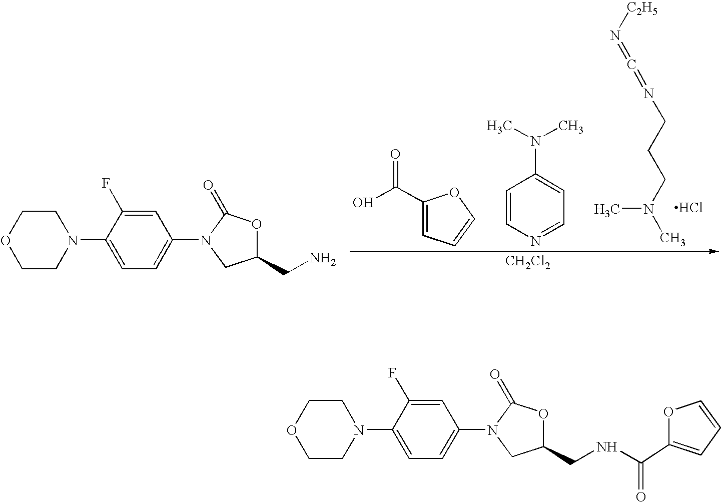

A solution of 57 mg (1.5 eq) of 2-furanoic acid, 21 mg (0.5 eq) of 4-(dimethylamino)pyridine (DMAP), 97 mg of 1-ethyl-3-(3′-dimethylaminopropyl)carbodiimide hydrochloride (EDCI.HCl, 1.5 eq) in 5 mL of dichloromethane (DCM) was stirred at room temperature under argon for 30 minutes. Then, 100 mg (1 eq) of N-[[(5S)-3-[3-fluoro-4-(4-morpholinyl)-phenyl]-2-oxo-5-oxazolidinyl]methyl]amine were added in 5 mL of DCM and stirring was continued for 12 hours when complete conversion of the starting amine was observed by TLC. The crude mixture was washed with 5% HOAc solution, saturated NaHCO₃ and brine. The combined organic layers were dried (MgSO₄) and concentrated in vacuum to afford 125 mg of N-[[(5S)-3-[3-fluoro-4-(4-morpholinyl)-phenyl]-2-oxo-5-oxazolidinyl]methyl]furan-2-yl-amide (Yield=95%).

¹H NMR (400 MHz, δ, ppm, CDCl₃): 3.05 (4H, m), 3.79 (2H, m), 3.86 (m, 5H), 4.05 (1H, t, J=8.8 Hz), 4.84 (1H, m), 6.49 (1H, dd, J=4.5 Hz), 6.81 (1H, t, J=5 Hz), 6.93 (1H, t, J=6.6 Hz), 7.06 (1H, m), 7.12 (1H, dd, J=3.2, 0.8 Hz), 7.4 (1H, m), 7.44 (1H, m).

HPLC (t, %): 6.99 min, 99%.

MS (ESI) m/z=390 (M+1)

### EXAMPLE 2

### N-[[(5S)-3-[3-fluoro-4-(4-morpholinyl)-phenyl]-2-oxo-5-oxazolidinyl]methyl]furan-2-yl-thioamide

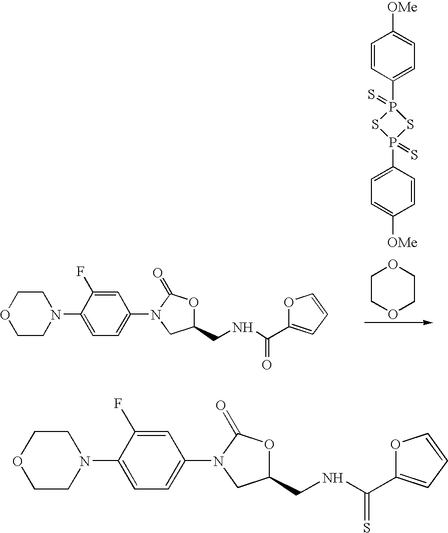

A solution of 87 mg of N-[[(5S)-3-[3-fluoro-4-(4-morpholinyl)-phenyl]-2-oxo-5-oxazolidinyl]methyl]furan-2-yl-amide, 271.3 mg (3 eq) of Lawesson's reagent in 4 mL of 1,4-dioxane was heated at 65° C. for 3 hours and at 100° C. for 1 h. The solvent was removed under reduced pressure and the crude was purified by column chromatography (Merck silica gel, DCM/MeOH 99/1) to afford 87 mg of the title product (Yield=96%).

HPLC (t, %): 11.3 min, 96%.

MS (ESI) m/z=406 (M+1)

### EXAMPLE 3

### N-[[(5S)-3-[3-fluoro-4-(4-morpholinyl)-phenyl]-2-oxo-5-oxazolidinyl]methyl]furan-3-yl-amide

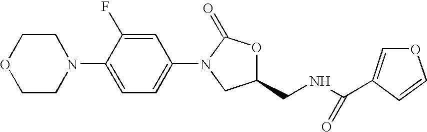

It was prepared following the same procedure as in Example 1, starting from 57 mg of 3-furanoic acid and 100 mg of N-[[(5S)-3-[3-fluoro-4-(4-morpholinyl)-phenyl]-2-oxo-5-oxazolidinyl]methyl]amine. After similar work-up, 125 mg were obtained corresponding to the desired N-[[(5S)-3-[3-fluoro-4-(4-morpholinyl)-phenyl]-2-oxo-5-oxazolidinyl]methyl]furan-3-yl-amide (Yield=95%).

HPLC (t, %): 7.76 min, 99%.

MS (ESI) m/z=390 (M+1).

### EXAMPLE 4

### N-[[(5S)-3-[3-fluoro-4-(4-morpholinyl)-phenyl]-2-oxo-5-oxazolidinyl]methyl]furan-3-yl-thioamide

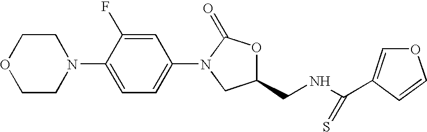

It was prepared following the same procedure as in Example 2, starting from 57 mg of N-[[(5S)-3-[3-fluoro-4-(4-morpholinyl)-phenyl]-2-oxo-5-oxazolidinyl]methyl]furan-3-yl-amide and 168.4 mg (4 eq) of Lawesson's reagent. The crude product was purified by column chromatography (silica gel, DCM/MeOH 99/1) to yield 53 mg of the title product (Yield=95%).

HPLC: 11.7 min, 99%.

MS (ESI) m/z=406 (M+1).

### EXAMPLE 5

### N-[[(5S)-3-[3-fluoro-4-(4′-acetyl-4-piperazinyl)phenyl]-2-oxo-5-oxazolidinyl]methyl]furan-2-yl-amide

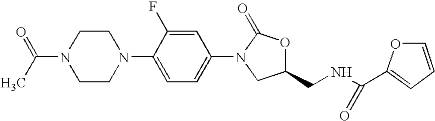

It was prepared following the same procedure as in Example 1, starting from 57 mg of 2-furanoic acid and 190 mg of N-[[(5S)-3-[3-fluoro-4-(4′-acetyl-4-piperazinyl)-phenyl]-2-oxo-5-oxazolidinyl]methyl]amine. The crude was worked up to give 60 mg of N-[[(5S)-3-[3-fluoro-4-(4-morpholinyl)-phenyl]-2-oxo-5-oxazolidinyl]methyl]furan-3-yl-amide (Yield=25%).

HPLC (t, %): 6.0 min, 94%.

MS (ESI) m/z=417 (M+1).

¹H NMR (400 MHz, δ, ppm, CDCl₃): 2.1 (3H, s), 2.98 (4H, m), 3.6 (2H, m), 3.80 (5H, m), 4.04 (1H, t, J=9.2 Hz), 4.83 (1H, m), 6.48 (1H, m), 6.86 (1H, t, J=9.2 Hz), 6.96 (NH), 7.04 (1H, m), 7.11 (1H, m), 7.40 (1H, m), 7.43 (1H, m).

### EXAMPLE 6

### N-[[(5S)-3-[3-fluoro-4-(4′-acetyl-4-piperazinyl)-phenyl]-2-oxo-5-oxazolidinyl]methyl]furan-3-yl-amide

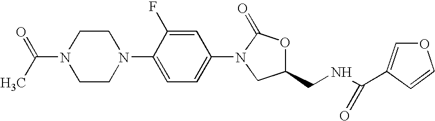

It was prepared following the same procedure as in Example 1, starting from 57 mg of 3-furanoic acid and 190 mg of N-[[(5S)-3-[3-fluoro-4-(4′-acetyl-4-piperazinyl)-phenyl]-2-oxo-5-oxazolidinyl]methyl]amine. The crude was worked up to give 80 mg of N-[[(5S)-3-[3-fluoro-4-(4-morpholinyl)-phenyl]-2-oxo-5-oxazolidinyl]methyl]furan-3-yl-amide (Yield=36%).

¹H NMR (400 MHz, δ, ppm, CDCl₃): 2.90 (2H, m), 2.96 (2H, m), 3.54 (2H, m), 3.65 (5H, m), 3.98 (1H, t, J=9.2 Hz), 4.78 (1H, m), 6.62 (1H, m), 6.80 (1H, t, J=9.2 Hz), 6.96 (2H, m), 7.34 (2H, m), 7.91 (1H, m).

HPLC: 6.4 min.

MS (ESI) m/z=431 (M+1).

### EXAMPLE 7

### N-[[(5S)-3-[3-fluoro-4-(4′-thioacetyl-4-piperazinyl)-phenyl]-2-oxo-5-oxazolidinyl]methyl]furan-3-yl-thioamide

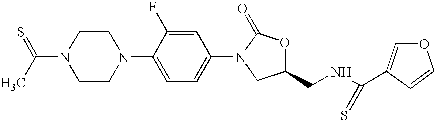

It was prepared following the same procedure as in Example 2, starting from 22 mg of N-[[(5S)-3-[3-fluoro-4-(4′-acetyl-4-piperazinyl)-phenyl]-2-oxo-5-oxazolidinyl]methyl]furan-3-yl-amide and 60 mg (3 eq) of Lawesson's reagent. The crude product was purified by column chromatography (silica get, DCM/MeOH 95/5) to yield 18 mg of the title product (Yield=79%).

HPLC: 12.9 min

MS (ESI) m/z=463 (M+1).

¹H NMR (400 MHz, δ, ppm, CDCl₃): 2.64 (3H, s), 3.05 (4H, m), 3.81 (3H, m), 4.06 (2H, m), 4.39 (3H, m), 5.00 (1H, m), 6.66 (1H, s), 6.83 (1H, t, J=9.2 Hz), 6.98 (1H, m), 7.36 (2H, m), 8.00 (1H, s), 8.14 (1H, NH).

### EXAMPLE 8

### N-[[(5S)-3-[3-fluoro-4-(4-thiomorpholinyl)-phenyl]-2-oxo-5-oxazolidinyl]methyl]furan-2-yl-amide

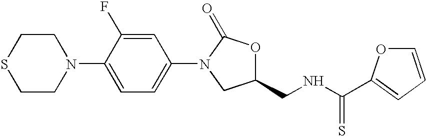

It was prepared following the same procedure as in Example 1, starting from 130 mg of 2-furanoic acid and 250 mg of N-[[(5S)-3-[3-fluoro-4-(4-thiomorpholinyl)-phenyl]-2-oxo-5-oxazolidinyl]methyl]amine. The crude was worked up to give 250 mg of the title compound (Yield=77%).

HPLC: 10.6 min

MS (ESI) m/z=406 (M+1).

¹H NMR (400 MHz, δ, ppm, CDCl₃): 2.77 (4H, m), 3.25 (4H, m), 3.96 (3H, m), 4.04 (1H, t, J=9.2 Hz), 4.83 (1H, m), 6.47 (1H, m), 6.89 (1H, t, J=9.6 Hz), 6.94 (NH), 7.03 (1H, m), 7.10 (1H, m), 7.38 (1H, dd, J=14.4, 2.8 Hz), 7.42 (1H, m).

### EXAMPLE 9

### N-[[(5S)-3-[3-fluoro-4-(4-thiomorpholinyl)-phenyl]-2-oxo-5-oxazolidinyl]methyl]furan-2-yl-thioamide

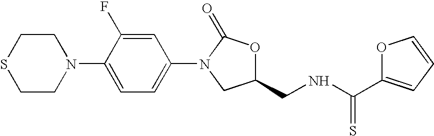

It was prepared following the same procedure as in Example 2, starting from 40 mg of N-[[(5S)-3-[3-fluoro-4-(4-thiomorpholinyl)-phenyl]-2-oxo-5-oxazolidinyl]methyl]furan-2-yl-amide and 200 mg (5 eq) of Lawesson's reagent. The crude product was purified by column chromatography (silica gel, DCM/MeOH 99/1) to yield 16 mg of the title product (Yield=39%).

HPLC: 13.9 min

MS (ESI) m/z=422 (M+1).

### EXAMPLE 10

### N-[[(5S)-3-[3-fluoro-4-(4-thiomorpholinyl)-phenyl]-2-oxo-5-oxazolidinyl]methyl]furan-3-yl-amide

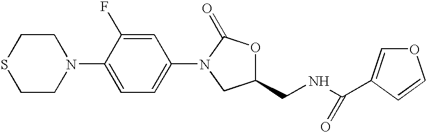

It was prepared following the same procedure as in Example 1, starting from 320 mg of 3-furanoic acid and 600 mg of N-[[(5S)-3-[3-fluoro-4-(4-thiomorpholinyl)-phenyl]-2-oxo-5-oxazolidinyl]methyl]amine. The crude was worked up to give 730 mg of the title compound (Yield=77%).

HPLC (t, %): 10.9 min, 98%.

MS (ESI) m/z=406 (M+1).

¹H NMR (400 MHz, δ, ppm, CDCl₃): 2.77 (4H, m), 3.24 (4H, m), 3.77 (3H, m), 4.03 (1H, t, J=8.8 Hz), 4.84 (1H, m), 6.67 (1H, m), 6.88 (1H, t, J=9.2 Hz), 7.00 (1H, m), 7.06 (NH), 7.34 (1H, m), 7.38 (1H, m), 7.96 (1H, m).

### EXAMPLE 11

### N-[[(5S)-3-[3-fluoro-4-(4-thiomorpholinyl)-phenyl]-2-oxo-5-oxazolidinyl]methyl]furan-3-yl-thioamide

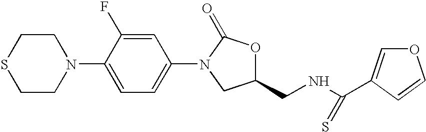

It was prepared following the same procedure as in Example 2, starting from 40 mg of N-[[(5S)-3-[3-fluoro-4-(4-thiomorpholinyl)-phenyl]-2-oxo-5-oxazolidinyl]methyl]furan-3-yl-amide and 160 mg (4 eq) of Lawesson's reagent. The crude product was purified by column chromatography (silica gel, hexane/ethylacetate 95/5) to yield 20 mg of the title product (Yield=48%).

HPLC: 14.4 min

MS (ESI) m/z=422 (M+1).

### EXAMPLE 12

### N-[[(5S)-3-[3-fluoro-4-(1-oxothiomorpholin-4-yl)-phenyl]-2-oxo-5-oxazolidinyl]methyl]furan-3-yl-amide

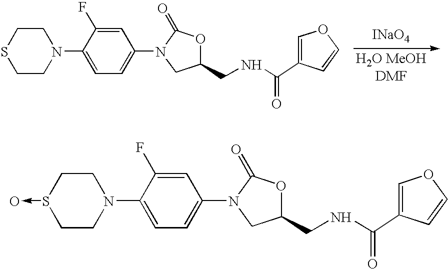

70 mg (1.05 eq) of sodium metaperiodate were dissolved in 1 mL of water and then cooled to 0° C. (ice bath). Next 130 mg (1 eq) of N-[[(5S)-3-[3-fluoro-4-(4-thiomorpholinyl)-phenyl]-2-oxo-5-oxazolidinyl]methyl]furan-3-yl-amide in 3.5 mL of methanol were added. 0.5 mL of dimethylformamide (DMF) were added to increase solubility. The reaction was stirred at 0° C. for 3 hours until TLC showed complete conversion of the starting material. The crude mixture was filtered to remove a white solid, which was further washed with DCM. The filtrate was transferred to a separatory funnel, the layers separated and the water layer further extracted with DCM. The organic layers were combined, dried over MgSO₄, filtered and concentrated under reduced pressure to yield 168 mg. This solid was purified by column chromatography (16 g of silica gel, DCM/MeOH in increasing polarity) to give 90 mg (Yield=68%) of the title compound.

HPLC (t, %): 5.04 min, 99.5%.

MS (ESI) m/z=422 (M+1).

¹H NMR (400 MHz, δ, ppm, CDCl₃): 2.97 (4H, m), 3.23 (2H, m), 3.75 (5H, m), 4.05 (1H, t, J=9.2 Hz), 4.85 (1H, m), 6.66 (1H, m), 6.85 (NH), 7.01 (1H, t, J=18 Hz), 7.05 (1H, m), 7.42 (2H, m), 7.97 (1H, m).

### EXAMPLE 13

### N-[[(5S)-3-[3-fluoro-4-(1,1-dioxothio-morpholin-4-yl)-phenyl]-2-oxo-5-oxazolidinyl]methyl]furan-3-yl-amide

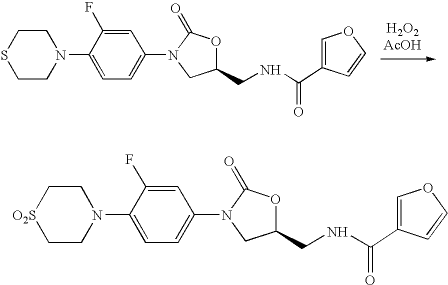

A solution of 120 mg (1 eq) of N-[[(5S)-3-[3-fluoro-4-(4-thiomorpholinyl)-phenyl]-2-oxo-5-oxazolidinyl]methyl]furan-3-yl-amide in 7 mL (1 eq) of acetic acid and 130 mL (4 eq) of H₂O₂ 30% was stirred under reflux for 2 hours. The solvent was evaporated under vacuum to give 118 mg of a reddish solid. This crude was purified by column chromatography (16 g of silica gel, DCM/MeOH in increasing polarity) yielding 24 mg (Yield=19%) of the title compound.

HPLC (t, %): 7.15 min, 90.7%.

MS (ESI) m/z=438 (M+1).

¹H NMR (400 MHz, δ, ppm, CDCl₃): 3.19 (4H, m), 3.56 (4H, m), 3.8 (3H, m), 4.06 (1H, t, J=9.2 Hz), 4.85 (1H, m), 6.48 (NH), 6.63 (1H, m), 6.98 (1H, t, J=9.2 Hz), 7.07 (1H, m), 7.45 (2H, m), 7.95 (1H, m).

### EXAMPLE 14

### Ethyl furan-2-carboximidate hydrochloride

To a cold (0° C.) solution of 1.3 g (14.2 mmol) of furan-2-carbonitrile in 10 mL of ethanol was passed hydrogen chloride gas (generated in situ from NaCl and H₂SO₄) for 20 hours. The solvent was evaporated under vacuum and the product recrystallized from ether to give 2.26 g of the title product (Yield=90%).

HPLC (t, %): 5.8 min, 97%.

¹H NMR (400 MHz, δ, ppm, CD₃OD): 1.56 (3H, t, J=7.2 Hz), 4.59 (4H, q, J=6.8 Hz), 6.83 (1H, dd, J=1.6 Hz, 3.6 Hz), 7.65 (1H, dd, 0.8 Hz, 3.6 Hz), 8.04 (1H, J=0.8 Hz, 1.6 Hz).

### EXAMPLE 15

### Ethyl furan-2-carboxycyanoimidate

A solution of 0.5 g (2.8 mmol) of ethyl furan-2-carboximidate hydrochloride and 0.59 g (14.2 mmol) of cyanamide in 4 mL of ethanol was heated at 40° C. under argon for 20 hours until TLC showed complete conversion. The ammonium chloride formed during the reaction was filtered off and the filtrate concentrated in vacuum to afford 0.888 g. This crude was dissolved in ethyl acetate and washed with water and brine. The combined organic layers were dried with MgSO₄ and concentrated in vacuum to give 0.360 g (Yield=77%) of a crystalline solid corresponding to the title compound.

HPLC (t, %): 7.9 min, 85%.

¹H NMR (400 MHz, δ, ppm, DMSO): 1.36 (3H, t, J=8 Hz), 4.41 (2H, q, J=7.2 Hz), 6.86 (1H, m), 7.74 (1H, m), 8.15 (1H, m).

FTIR (film, v, cm⁻¹): 2200.

### EXAMPLE 16

### N-[[(5S)-3-[3-fluoro-4-(4-morpholinyl)-phenyl]-2-oxo-5-oxazolidinyl]methyl]furan-3-yl-cyanoamidine

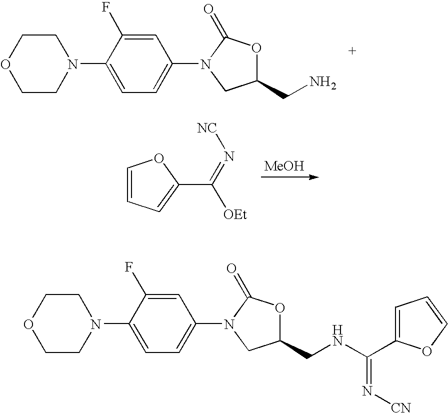

A solution of 50 mg (0.17 mmol) of N-[[(5S)-3-[3-fluoro-4-(4-morpholinyl)-phenyl]-2-oxo-5-oxazolidinyl]methyl]amine and 83 mg (0.5 mmol) of ethyl furan-3-carboxycyanoimidate in 5 mL of methanol was refluxed under argon overnight. The reaction mixture, which contained a white precipitate, was filtered. The solid was washed with methanol and dried under vacuum to give 51 mg (Yield=73%) of the desired compound.

HPLC (t, %): 8.7 min, 100%.

MS (ESI) m/z=414 (M+1).

¹H NMR (400 MHz, δ, ppm, DMSO): 2.89 (4H, m), 3.71 (2H, m), 3.76 (4H, m), 3.85 (1H, dd, J=6.4, 9.6 Hz), 4.17 (1H, t, J=8.8 Hz), 4.94 (1H, m), 6.84 (1H, m), 7.09 (1H, t, J=9.6 Hz), 7.21 (1H, m), 7.49 (1H, m), 7.71 (1H, m). 8.07 (1H, m). 9.45 (NH).

### EXAMPLE 17

### N-[[(5S)-3-[3-fluoro-4-(4′-acetyl-4-piperazinyl)-phenyl]-2-oxo-5-oxazolidinyl]methyl]furan-3-yl-cyanoamidine

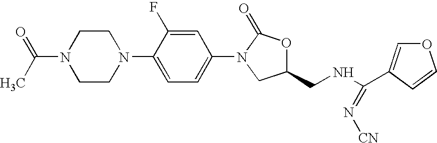

It was prepared following the same procedure as in Example 16, starting from 50 mg (0.15 mmol) of N-[[(5S)-3-[3-fluoro-4-(4′-acetyl-4-piperazinyl)-phenyl]-2-oxo-5-oxazolidinyl]methyl]amine and 73.2 mg (0.44 mmol) of ethyl furan-2-carboxycyanoimidate. After refluxing overnight a complete conversion was observed by TLC. The crude was left at room temperature over the weekend and a white precipitate was obtained. The solid was filtered, washed with methanol and dried under vacuum. ¹H NMR showed an impurity which was purified by column chromatography (silica gel, DCM/MeOH, 95:5) to give 44 mg of the desired product.

HPLC (t, %): 7.3 min, 99%.

MS (ESI) m/z=455 (M+1).

¹H NMR (400 MHz, δ, ppm, CDCl₃): 2.14 (3H, s), 3.01 (2H, m), 3.085 (2H, m), 3.62 (2H, m), 3.77 (4H, m), 4.03 (1H, m), 4.13 (1H, t, J=9.2 Hz), 4.91 (1H, m), 6.65 (1H, dd, J=2, 3.6 Hz), 6.82 (NH), 6.91 (1H, t, J=8.9 Hz), 7.05 (1H, m), 7.46 (1H, dd, J=2.4, 14 Hz), 7.56 (1H, m), 8.045 (1H, d, J=4 Hz).

### EXAMPLE 18

### N-[[(5S)-3-[3-fluoro-4-(4-thiomorpholinyl)-phenyl]-2-oxo-5-oxazolidinyl]methyl]furan-3-yl-cyanoamidine

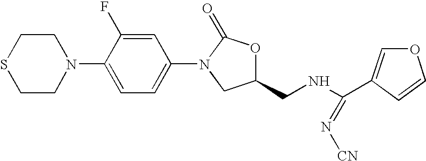

It was prepared following the same procedure as in Example 16, starting from 50 mg (0.16 mmol) of N-[[(5S)-3-[3-fluoro-4-(4-thiomorpholinyl)-phenyl]-2-oxo-5-oxazolidinyl]methyl]amine and 79.2 mg (0.48 mmol) of ethyl furan-3-carboxycyanoimidate. After refluxing overnight a complete conversion was observed by TLC. Because the product did not precipitate, 0.141 g (0.32 mmol) of tris-(2 aminoethyl)amine polystyrene was added and kept under reflux overnight when the excess of cyanoimidate disappeared by TLC. The resin was filtered off and the filtrate was concentrated under vacuum to give 62 mg of the title product.

HPLC (t, %): 11.6 min, 99%.

MS (ESI) m/z=430 (M+1).

¹H NMR (400 MHz, δ, ppm, CD₃OD): 2.81 (4H, m), 3.30 (2H, m), 3.85 (3H, m), 4.22 (1H, t, J=9.2 Hz), 5.01 (1H, m), 6.77 (1H, m), 7.09 (1H, t, J=8.8 Hz), 7.19 (1H, m), 7.49 (1H, dd, J=2.4, 14 Hz), 7.84 (2H, m).

### EXAMPLE 19

### N-[[(5S)-3-[3-fluoro-4-(1-oxothiomorpholin-4-yl)-phenyl]-2-oxo-5-oxazolidinyl]methyl]furan-3-yl-cyanoamidine

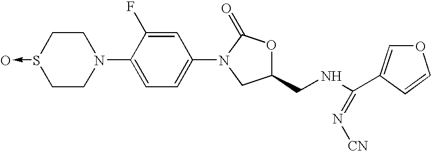

31.4 mg (1.05 eq) of sodium metaperiodate were dissolved in 0.5 mL of water and then cooled to 0° C. (ice bath). Next, 60 mg (1 eq) of N-[[(5S)-3-[3-fluoro-4-(4-thiomorpholinyl)-phenyl]-2-oxo-5-oxazolidinyl]methyl]furan-3-yl-cyanoamidine in 2 mL of methanol were added and a white precipitate was formed. The reaction was stirred at 0° C. for 3 hours and overnight at room temperature until TLC showed complete conversion of the starting material. The crude mixture was filtered to remove a white solid, which was further washed with DCM. The filtrate was transferred to a separatory funnel, the layers separated and the water layer further extracted with DCM.

The organic layers were combined, dried over MgSO₄, filtered and concentrated under reduced pressure to yield 62 mg. This solid was purified by column chromatography (silica gel, DCM/MeOH in increasing polarity) to give 56 mg (Yield=90%) of the title compound.

HPLC (t, %): 5.9 min, 100%.

MS (ESI) m/z=446 (M+1).

¹H NMR (400 MHz, δ, ppm, CD₃OD): 2.98 (4H, m), 3.25 (2H, m), 3.76 (4H, m), 4.04 (1H, m), 4.13 (1H, t, J=9.2 Hz), 4.92 (1H, m) 6.65 (1H, m), 6.83 (NH), 7.05 (2H, m), 7.48 (1H, m), 7.56 (1H, m), 8.045 (1H, d, J=4 Hz).

The compounds of Table 1 below were prepared following same procedure as in Example 1:

TABLE I

| | | HPLC t(min), | MS(ESI) |
|---|---|---|---|
| Ex. | Structure | (%) | m/z |
| 20 | 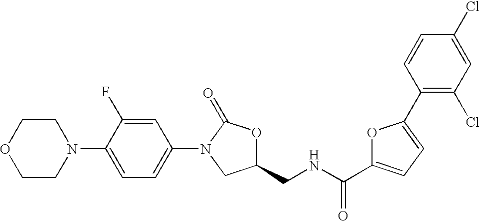 | 14.8 (99%) | 534-536 |
| 21 | 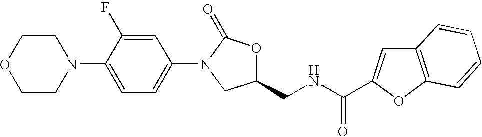 | 10.9 (98%) | 440 |
| 22 | 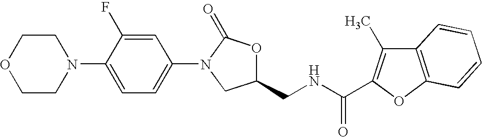 | 12.6 (98%) | 454 |
| 23 | 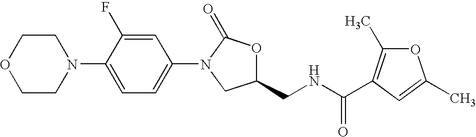 | 10.0 (100%) | 418 |
| 24 | 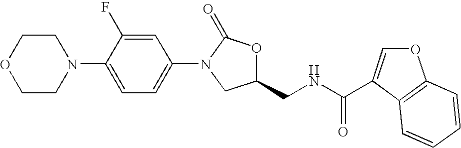 | 11.0 (87%) | 440 |
| 25 | 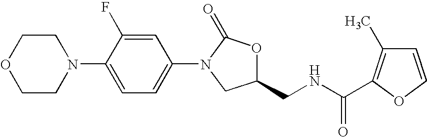 | 9.0 (100%) | 404 |
| 26 | 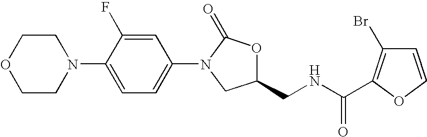 | 9.1 (100%) | 468-470 |
| 27 | 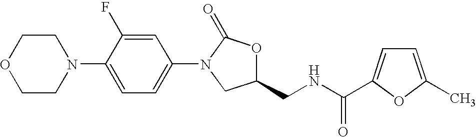 | 8.4 (100%) | 404 |
| 28 | 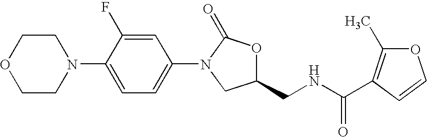 | 9.1 (100%) | 404 |
| 29 | 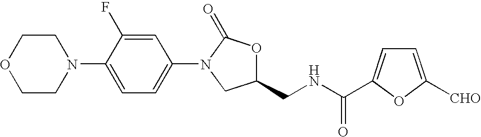 | 7.1 (100%) | 418 |
| 30 | 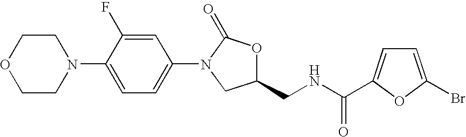 | 9.8 (97%) | 468-470 |
| 31 | 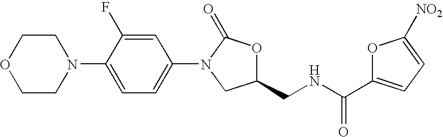 | 8.9 (100%) | 435 |
| 32 | 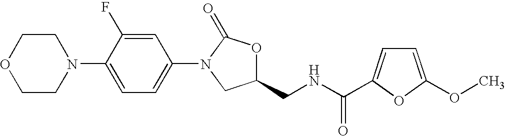 | 8.05 (97%) | 420 |
| 33 | 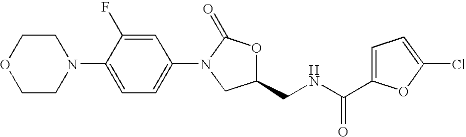 | 9.5 (97%) | 424-426 |
| 34 | 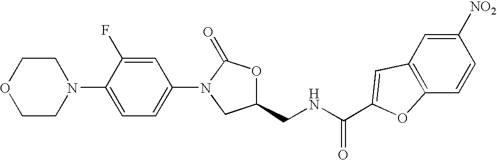 | 5.7 (98%) | 485 |
| 35 | 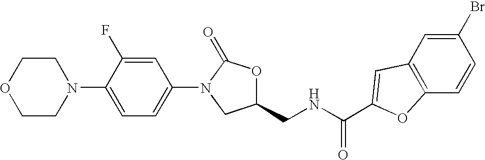 | 6.6 (96%) | 518-520 |
| 36 | 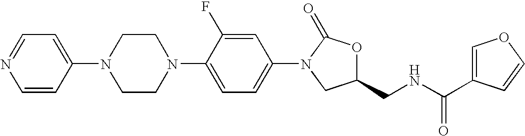 | 8.0 (95%) | 483 (M + 18) |
| 37 | 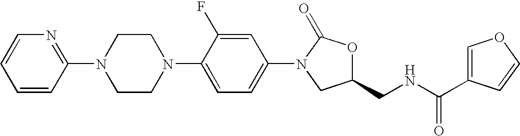 | 11.3 (70%) | 466 |

### EXAMPLE 38

### N-[[(5S)-3-[3-fluoro-4-(1-oxothiomorpholin-4-yl)-phenyl]-2-oxo-5-oxazolidinyl]methyl]furan-3-yl-thioamide

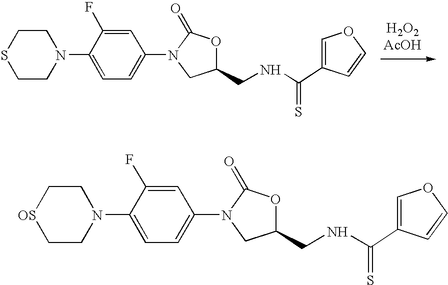

A solution of 270 mg (1 eq) of N-[[(5S)-3-[3-fluoro-4-(4-thiomorpholinyl)-phenyl]-2-oxo-5-oxazolidinyl]methyl]furan-3-yl-thioamide (Example 11) in 15 mL of acetic acid and 600 μL (8 eq) of H₂O₂ 30% was stirred under reflux for 2 hours. The solvent was evaporated under vacuum and washed with a saturated solution of NaHCO₃ to give 360 mg of a crude product. This crude was purified by column chromatography (10 g of silica gel, DCM/MeOH in increasing polarity up to 95/5) yielding 104 mg (Yield=39%) of the title compound.

HPLC (t, %): 8.6 min, 96%.

MS (ESI) m/z=438 (M+1).

¹H NMR (400 MHz, δ, ppm, CDCl₃): 2.98 (4H, m), 3.23 (2H, m), 3.7 (2H, m), 3.86 (1H, m), 4.13 (2H, m), 4.4 (1H, m), 5.06 (1H, m), 6.75 (1H, m), 7.02 (1H, m), 7.42 (2H, m), 8.49 (NH).

### EXAMPLE 39

### N-[(5S)-[3-[3-fluoro-4-[(N-t-butoxycarbonyl)piperazin-1-yl]phenyl]-2-oxo-5-oxazolidinylmethyl]amine

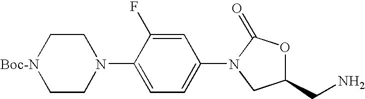

This compound can be obtained by two procedures:

Procedure A: To a (S)-[3-[3-fluoro-4-[N-t-butoxycarbonylpiperazin-1-yl]phenyl]-2-oxooxazolidin-5-ylmethyl]azide (27.6 mmol) in EtOAc 10% Pd/C (6.4 g) was added and the reaction was allowed to stir at ambient temperature under H₂ balloon condition. The reaction was complete by TLC, the mixture was filtered through Celite and concentrated under vacuum. The purity of the crude product is higher of 95% but must be kept under argon to avoid amine oxidation.

Procedure B: To a (5S)-[3-[3-fluoro-4-[N-t-butoxycarbonylpiperazin-1-yl]phenyl]-2-oxooxazolidin-5-ylmethyl]alcohol (74.1 g, 0.19 mol) and triethylamine (36 mL, 0.26 mol) in DCM (750 mL) was added slowly 3-nitrobenzensulfonyl chloride (55.6 g, 0.25 mol). The reaction was stirred for 24 hours, then washed with water (500 mL), dried and evaporated to give (5S)-[3-[3-fluoro-4-[N-t-butoxycarbonylpiperazin-1-yl]phenyl]-2-oxooxazolidin-5-ylmethyl]nosylate (116 g) containing some unreacted 3-nitro-benzenesulfonyl chloride. To a solution of this previous nosylate (115 g) in acetonitrile (2 L) was added concentrated ammonia (d=0.88, 100 mL) and the reaction mixture was heated to 40° C. for 3 hours. A second portion of ammonia (500 mL) was added and the mixture maintained at 40° C. overnight. A third portion of ammonia (500 mL) was added, followed 8 hours later by a final portion of ammonia (500 mL) and another overnight stir. The cooled reaction mixture was split into two portions, and each half diluted with water (1 L) and extracted with DCM (2×1 L). The combined DCM extracts were dried and evaporated to give 71.4 g of the desired product.

¹H NMR (400 MHz, δ, ppm, CD3OD): 1.48 (9H, s), 2.96 (6H, m), 3.57 (4H, m), 3.81 (1H, m), 4.09 (1H, t, J=16 Hz), 4.7 (1H, m), 7.05 (1H, t, J=8 Hz), 7.19 (1H, m), 7.51 (1H, dd, J=2.4, 14 Hz).

HPLC (t, %): 4.8 min, 97%.

MS (ESI) m/z=395 (M+1).

### EXAMPLE 40

### N-[[(5S)-[3-[3-fluoro-4-[(N-t-butoxycarbonyl)piperazin-1-yl]phenyl]-2-oxo-5-oxazolidinylmethyl]furan-3-yl-amide

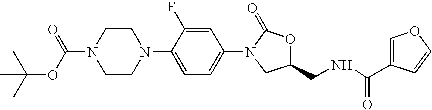

A mixture of 3-furanoic acid (2.13 g, 12.72 mmol), EDCI (4.86 g, 25.5 mmol), DMAP (0.3 g, 2.5 mmol) and DCM (50 mL) was stirred for 30 minutes then a solution of N-[(5S)-[3-[3-fluoro-4-[(N-t-butoxycarbonyl)piperazin-1-yl]phenyl]-2-oxo-5-oxazolidinylmethyl]amine (5 g, 12.7 mmol) in 50 mL DCM was added. After stirring overnight, the mixture was washed with 5% acetic acid solution, saturated NaHCO₃, and finally brine. The solvent was evaporated under reduced pressure to give 5.1 g of desired product (93% yield). The crude mixture is purified by column chromatography eluting with DCM/MeOH 98/2, to give the title product in a 95% purity by HPLC.

¹H NMR (400 MHz, δ, ppm, DMSO): 1.4 (9H, s), 2.89 (4H, m), 3.45 (4H, m), 3.55 (2H, m), 3.78 (1H, m), 4.12 (1H, t, J=9 Hz), 4.78 (1H, m), 6.85 (1H, m), 7.06 (1H, t, J=9.2 Hz), 7.17 (2H, m), 7.47 (2H, m), 7.71 (1H, m), 8.19 (1H, s), 8.55 (NH).

HPLC: 6.3 min.

MS (ESI) m/z=489 (M+1).

### EXAMPLE 41

### N-[[(5S)-[3-[3-fluoro-4-(piperazin-1-yl)phenyl]-2-oxo-5-oxazolidinylmethyl]furan-3-yl-amide

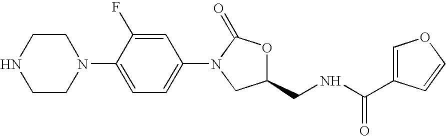

To a solution of Boc-protected derivative of example 40 (1 g) in DCM (15 mL) at 0° C. was added a 15 mL of trifluoroacetic acid over 10 minutes. After 15 minutes, the mixture was allowed to warm up to room temperature and stirred for one hour. The solvent was removed under reduced pressure and the residue dissolved in water basified with NaHCO₃ to pH=8.9. Part of the product is precipitated from this aqueous solution and the solid separated by filtration. The basic solution is further extracted with DCM. The organic extracts were dried and the solvent removed under reduced pressure to give more product as a white solid. Both solids correspond to the title product in a 99% purity by HPLC.

¹H NMR (400 MHz, δ, ppm, DMSO): 2.83 (8H, m), 3.55 (2H, t, J=4 Hz), 3.78 (1H, m), 4.11 (1H, t, J=9 Hz), 4.78 (1H, m), 6.85 (1H, m), 7.02 (1H, t, J=9.2 Hz), 7.15 (2H, m), 7.44 (2H, m), 7.71 (1H, m), 8.19 (1H, s), 8.58 (NH).

HPLC: 2.6 min.

MS (ESI) m/z=389 (M+1).

### EXAMPLES 42-46

(Table 2 below) were prepared following the same general procedure. The appropriate acid (0.31 mmol), EDCI.HCl (0.5 mmol), DMAP (0.13 mmol) and DMF (3 mL) were stirred for 30 minutes, then compound of Example 41 (100 mg, 0.26 mmol) was added. The mixture was stirred for ca. 24 hours at room temperature and 2 h at 60° C. The mixture was washed with 5% acetic acid solution (3 mL), saturated NaHCO₃ solution (3 mL), and finally brine (3 mL). The organic phase was dried and the solvent was evaporated under reduced pressure to give the solid product which was finally washed with ethyl ether.

### EXAMPLES 47-49

(Table 2 below) were prepared following the same general procedure. The appropriate acid (0.31 mmol), EDCI.HCl (0.5 mmol), DMAP (0.13 mmol) and DMF (3 mL) were stirred for 30 minutes, then compound of Example 41 (100 mg, 0.26 mmol) was added. The mixture was stirred for ca. 24 hours at room temperature and 2 h at 60° C. To improve conversion a further equivalent of EDCI.HCl was added and the solution kept at 60° C. for 2 h. The crude mixture was washed with 5% acetic acid solution (2 mL), saturated K₂CO₃ solution (2 mL), and finally brine (2 mL). The organic phase was dried and the solvent was evaporated under reduced pressure to give the product, which was subsequently purified by trituration with ethyl ether.

### EXAMPLES 50-52

(Table 2 below) were prepared following the same general procedure. The appropriate acid (0.39 mmol), EDCI.HCl (0.39 mmol), DMAP (0.13 mmol) and DMF (3 mL) were stirred for 30 minutes, then compound of Example 41 (100 mg, 0.26 mmol) was added. The mixture was stirred for ca. 64 hours at room temperature. The crude mixture was washed with 5% acetic acid solution (2 mL), saturated K₂CO₃ solution (2 mL), and finally brine (2 mL). The organic phase was dried and the solvent was evaporated under reduced pressure to give the product, which was subsequently purified by trituration with ethyl ether. In the case of compound 53, only this was purified by preparative HPLC (column symmetry C18, 7 μm, 19×150 mm; mobile phase: t=0-12 min 2% acetonitrile+98% ammonium formate buffer (pH=5.22); t=22-5 min 60% acetonitrile+40% ammonium formate buffer; t=30 min 2% acetonitrile+98% ammonium formate buffer).

TABLE 2

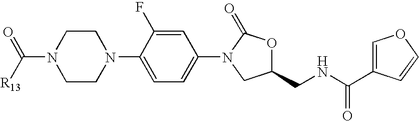

| | | | HPLC t(min), | |
|---|---|---|---|---|
| Ex. | R₁₃ | LC Purity | (%) | MS(ESI) m/z |
| 42 | 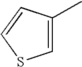 | 98 | 4.83 | 499 |
| 43 | 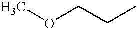 | 99 | 3.5 | 475 |
| 44 | 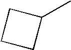 | 99 | 4.77 | 471 |
| 45 | 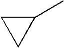 | 98 | 4.14 | 457 |
| 46 | 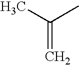 | 98 | 4.16 | 457 |
| 47 | 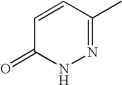 | 99 | 2.83 | 511 |
| 48 | 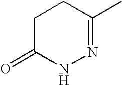 | 86 | 3.11 | 513 |
| 49 | 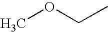 | 96 | 3.21 | 461 |
| 50 | 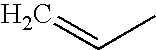 | 97 | 3.74 | 443 |
| 51 | 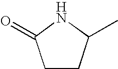 | 97 | 2.68 | 500 |
| 52 | 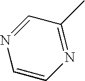 | 96 | 6.65 | 495 |

### EXAMPLE 53

### N-[[(5S)-3-[3-fluoro-4-(4′-benzyloxyacetyl-4-piperazinyl)-phenyl]-2-oxo-5-oxazolidinyl]methyl]furan-3-yl-amide

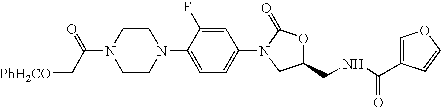

To a solution of 100 mg of compound of example 41 in 10 mL of DCM at 0° C. was added 56 μL of benzyloxyacetyl chloride dropwise. After 2 hours, the mixture was allowed to warm up to room temperature and stirred at room temperature overnight. The crude mixture was washed with water and the aqueous phase further extracted with DCM. The organic extracts were dried over MgSO₄ and solvent removed under reduced pressure to give 178 mg of an oily product. The crude was subsequently purified by trituration with ethyl ether to give 113 mg of a solid in acceptable purity.

1H NMR (400 MHz, δ, ppm, DMSO): 2.94 (4H, m), 3.55 (6H, m), 3.78 (1H, m), 4.12 (1H, t, J=9 Hz), 4.23 (2H, s), 4.53 (2H, s), 4.78 (1H, m), 6.85 (1H, m), 7.04 (1H, t, J=9 Hz), 7.15 (1H, m), 7.33 (5H, m), 7.48 (1H, m), 7.71 (1H, m), 8.19 (1H, m), 8.56 (NH).

HPLC: 7.74 min.

MS (ESI) m/z=537 (M+1).

### EXAMPLE 54

### N-[[(5S)-3-[3-fluoro-4-(4′-hydroxyacetyl-4-piperazinyl)-phenyl]-2-oxo-5-oxazolidinyl]methyl]furan-3-yl-amide

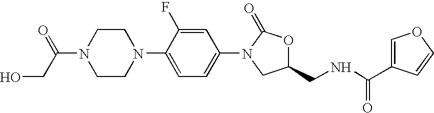

A solution of 97 mg of example 53 with 27 mg of Pd/C 10% in 10 mL DCM/MeOH 33% (v/v) was stirred at room temperature under hydrogen overnight. The crude was filtered through Celite and evaporated, and further purified by preparative HPLC.

HPLC: 6.2 min.

MS (ESI) m/z=447 (M+1).

### EXAMPLE 55

### Determination of Biological Data

(a) Antibacterial Activity

MICs were determined by using a standard microdilution method according to The National Committee for Clinical Laboratory Standards (NCCLS), 5^{th} Approved standard M7-A5, 2001, Wayne, Pa., USA.

All compounds were tested against Gram-positive and Gram-negative bacteria showing relevant different susceptibility and resistance specifications. The used microorganisms were selected from laboratory reference bacteria and from clinical isolates.

The tested concentrations were double dilutions from 0.06 μg/mL to 128 μg/mL in 96-well microtiter plates.

The microorganisms used in the study were:

Aerobic Gram-positive bacteria, consisting of Staphylococcus aureus, Staphylococcus epidermidis, Enterococcus faecalis, Enterococcus faecium and Streptococcus pneumoniae; and Moraxella catarrhalis, a Gram-negative bacterium, which is relevant to respiratory infections; it is also called fastidious because of its growing requirements.

MICs were determined in the Brucella blood medium supplemented for the anaerobic strains, and in the Mueller-Hinton culture medium (cation-adjusted) for the aerobic bacteria.

The tested compounds were dissolved in DMSO, and were diluted as far as 2560 μg/mL with the different media according to the specific requirements for each group of strains.

The 96-well sealed microtiter plates containing bacteria were incubated in different laboratory conditions depending on the nature of the microorganism. Thus, the aerobic bacteria were incubated during 16-24 h at 35° C. and the so-called fastidious bacteria, such as M. catarrhalis and S. pneumoniae, during 20-24 h at 35° C. in a microaerobiotic atmosphere containing 5% CO₂ (Anaerocult C, MERCK).

(b) In Vitro MAO-A and MAO-B Enzymatic Activity

MAO-A and MAO-B enzymatic activities were measured using membranes obtained from SF9 cells expressing either human MAO-A or human MAO-B (Gentest, BD, USA). Assays were done in blank 96-well microtiter plates using kynuramine as substrate and measuring the formation of 4-hydroxyquinoline by fluorescence at 340 nm/465 nm. Briefly, membranes with MAO-A (0.006 mg/mL protein) and MAO-B (0.015 mg/mL protein) were incubated with kynuramine, 30 μM, at 370 for 40 min in the presence of the compound in a final volume of 200 μL. Reactions were stopped by adding NaOH 2N and the reaction product, 4-hydroxyquinoline, was determined by fluorometry using a Tecan Ultra reader.

A low Kᵢ value indicates that the tested inhibitor possesses a tight binding ability to MAO enzyme, thus, it is a strong MAO inhibitor.

Antibacterial activity and MAO-A and MAO-B enzymatic activities are shown in Tables 3 and 4 respectively.

TABLE 3
In vitro activities against bacteria, MIC (μg/mL)

| | | Ex. | Ex. | Ex. | Ex. | |
|---|---|---|---|---|---|---|
| Organism | Ex. 2 | 4 | 7 | 9 | 11 | Linezolid |
| S. aureus ATCC 25923 MS 319 | 8.00 | 1.00 | 0.50 | 2.00 | 2.00 | 2.00 |
| S. aureus ATCC 43300 MR 214 | 2.00 | 1.00 | 0.50 | 4.00 | 1.00 | 1.00 |
| S. epidermidis | 2.00 | 1.00 | 0.50 | 4.00 | 1.00 | 1.00 |
| ATCC 12228 MR 11 | | | | | | |
| S. pneumoniae ATCC 49619 PR 215 | 2.00 | 1.00 | 0.50 | 4.00 | 1.00 | 2.00 |
| E. faecalis ATCC 29212 53 | 2.00 | 1.00 | 0.13 | 2.00 | 1.00 | 0.50 |
| E. faecalis ATCC 51575 MDR 311 | 2.00 | 1.00 | 0.06 | 2.00 | 1.00 | 0.50 |
| E. faecium ATCC 51559 MDR 312 | 1.00 | 2.00 | 0.50 | 8.00 | 1.00 | 1.00 |
| Moraxella catarrhalis HCl-78 259/339 | 2.00 | 1.00 | 0.25 | — | 4.00 | 2.00 |

TABLE 4
Inhibitory activity of human MAO, Ki (μmol)

| | | | | | | Line- | |
|---|---|---|---|---|---|---|---|
| | Ex. 2 | Ex. 4 | Ex. 7 | Ex. 9 | Ex. 11 | zolid | Toloxatone |
| MAO-A | 169.7 | ≧250 | ≧200 | 47.5 | ≧250 | 7.00 | 1.50 |
| MAO-B | 5.3 | 2.4 | 2.8 | 1.0 | 0.6 | 0.48 | 90 |

### EXAMPLE 56

### Pharmaceutical Compositions

The following illustrates representative pharmaceutical compositions containing a compound of formula (I) or a pharmaceutically acceptable salt thereof for antimicrobial use in human or animals:

| Tablet 1 | mg/tablet |
|---|---|
| Active ingredient | 100 |
| actose | 179 |
| Croscarmellose sodium | 12 |
| Polyvinylpyrrolidone | 6 |
| Magnesium stearate | 3 |

| Tablet 2 | mg/tablet |
|---|---|
| Active ingredient | 50 |
| Lactose | 229 |
| Croscarmellose sodium | 12 |
| Polyvinylpyrrolidone | 6 |
| Magnesium stearate | 3 |

| Tablet 3 | mg/tablet |
|---|---|
| Active ingredient | 1 |
| Lactose | 92 |
| Croscarmellose sodium | 4 |
| Polyvinylpyrrolidone | 2 |
| Magnesium stearate | 1 |

| Capsule | mg/capsule |
|---|---|
| Active ingredient | 10 |
| Lactose | 389 |
| Croscarmellose sodium | 100 |
| Magnesium stearate | 1 |

| Injection | 50 mg/mL |
|---|---|
| Active ingredient | 5.0% w/v |
| Isotonic aqueous solution | to 100% |

Buffers, pharmaceutically acceptable co-solvents such as polyethylene glycol, polypropylene glycol, glycerol or ethanol or chelating agents, may be used to aid formulation.

The above formulations may be prepared by well-known conventional procedures in the pharmaceutical art. The tablets 1-3 may be enteric coated by conventional means, for example to provide a coating of cellulose acetate phthalate.

1. A compound of the structural formula (I) or a pharmaceutically acceptable salt thereof, 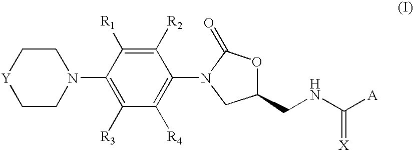
wherein:
—R₁, —R₂, —R₃ and —R₄ are radicals independently selected from hydrogen, F and Cl;
-A is a radical selected from the group consisting of 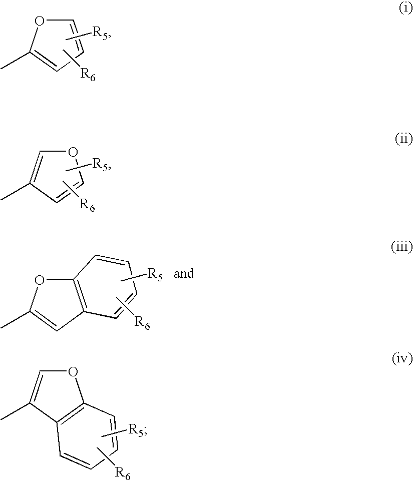
—R₅ and —R₆ are radicals independently selected from the group consisting of hydrogen, F, Cl, Br, —NO₂, —CN, —COR₇, —CSR₇, —SO₂R₇, —OCOR₇, alkyl(C₁-C₆), haloalkyl(C₁-C₆), cycloalkyl(C₃-C₆), alkenyl(C₂-C₆), alkynyl(C₂-C₆), alkoxyl(C₁-C₆), alkoxyalkyl(C₁-C₆), —NH-alkyl(C₁-C₆), —N-dialkyl(C₁-C₆), phenyl optionally substituted and heteroaryl optionally substituted; or —R₅ and —R₆ taken together form an optionally substituted benzo-fused ring;
—R₇ is a radical selected from the group consisting of hydrogen, alkyl(C₁-C₆), cycloalkyl(C₃-C₆), alkenyl(C₂-C₆), alkynyl(C₂-C₆), alkoxyl(C₁-C₆), alkoxyalkyl(C₁-C₆), hydroxyalkyl(C₁-C₆), —NH-alkyl(C₁-C₆), —N-dialkyl(C₁-C₆), phenyl optionally substituted and heteroaryl optionally substituted;
X is selected from O, S, NR₈ and CR₈R₉;
—R₈ and —R₉ are radicals independently selected from the group consisting of hydrogen, —CN, —COR₁₀, —SO₂R₁₀, alkyl(C₁-C₆), haloalkyl(C₁-C₆), cycloalkyl(C₃-C₆), alkenyl(C₂-C₆), alkynyl(C₂-C₆), alkoxyl(C₁-C₆), alkoxyalkyl(C₁-C₆), —NH-alkyl(C₁-C₆), —N-dialkyl(C₁-C₆), phenyl optionally substituted and heteroaryl optionally substituted;
—R₁₀ is a radical selected from the group consisting of hydrogen, alkyl(C₁-C₆), -haloalkyl(C₁-C₆), cycloalkyl(C₃-C₆), alkenyl(C₂-C₆), alkynyl(C₂-C₆), alkoxyalkyl(C₁-C₆), phenyl optionally substituted and heteroaryl optionally substituted;
—Y— is a biradical selected from O, S, SO, SO₂, NO, NR₁₁, and CR₁₁R₁₂;
—R₁₁ and —R₁₂ are a radical independently selected from the group consisting of hydrogen, —(CHR₁₃)ₙR₁₄, —CN, —COR₁₃, —CSR₁₃, —COOR₁₃, —CSOR₁₃, —CONR₁₃R₁₄, —CSNR₁₃R₁₄, —CON(R₁₅)N(R₁₄)R₁₃, —SO₂R₁₃, —SO₂OR₁₃, —SO₂NR₁₃R₁₄, alkyl(C₁-C₆), haloalkyl(C₁-C₆), cycloalkyl(C₃-C₆), alkenyl(C₂-C₆), alkynyl(C₂-C₆), alkoxyalkyl(C₁-C₆), phenyl optionally substituted and heteroaryl optionally substituted;
n is selected from 0 and 1;
—R₁₃ and —R₁₄ are a radical independently selected from the group consisting of hydrogen, —COR₁₅, —CSR₁₅, —SO₂R₁₅, alkyl(C₁-C₆), cycloalkyl(C₃-C₆), alkenyl(C₂-C₆), alkynyl(C₂-C₆), alkoxyl(C₁-C₆), alkoxyalkyl(C₁-C₆), hydroxyalkyl(C₁-C₆), dihydroxyalkyl(C₁-C₆), phenyl optionally substituted, 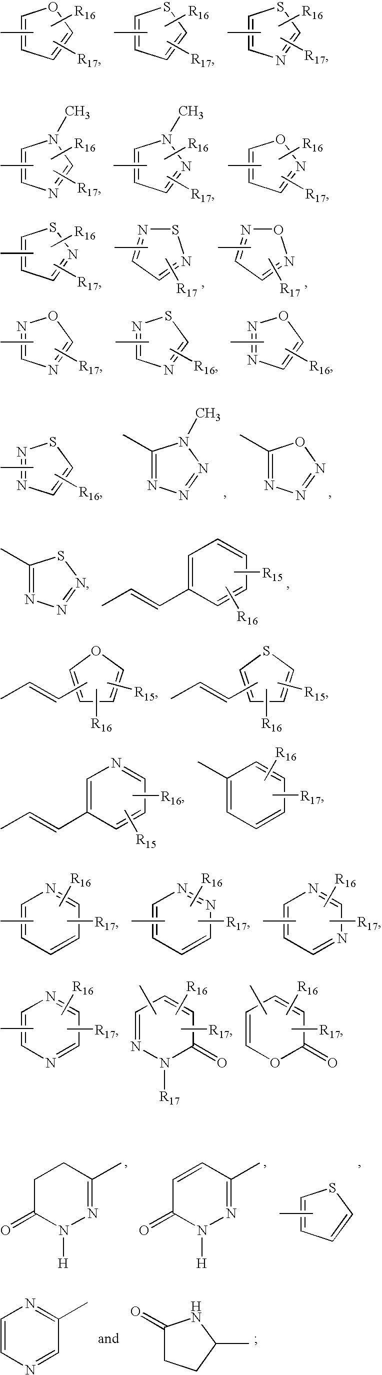
—R₁₅ is a radical selected from the group consisting of hydrogen, alkyl(C₁-C₆), cycloalkyl(C₃-C₆), alkenyl(C₂-C₆), alkynyl(C₂-C₆), alkoxyl(C₁-C₆), alkoxyalkyl(C₁-C₆), hydroxyalkyl(C₁-C₆), phenyl optionally substituted and heteroaryl optionally substituted;
—R₁₆ and —R₁₇ are radicals independently selected from the group consisting of F, Cl, Br, —NO₂, —CN, —COR₁₈, —CONR₁₈R₁₉, —SO₂R₁₈, —SO₂NR₁₈R₁₉, alkyl(C₁-C₆), haloalkyl(C₁-C₆), cycloalkyl(C₃-C₆), alkenyl(C₂-C₆), alkynyl(C₂-C₆), alkoxyl(C₁-C₆), alkoxyalkyl(C₁-C₆), phenyl optionally substituted and heteroaryl optionally substituted; and
—R₁₈ and —R₁₉ are radicals independently selected from the group consisting of hydrogen, alkyl(C₁-C₆), haloalkyl(C₁-C₆), cycloalkyl(C₃-C₆), alkenyl(C₂-C₆), alkynyl(C₂-C₆), alkoxyl(C₁-C₆), alkoxyalkyl(C₁-C₆), phenyl optionally substituted and heteroaryl optionally substituted.

2. A compound of the structural formula (I) or a pharmaceutically acceptable salt thereof: 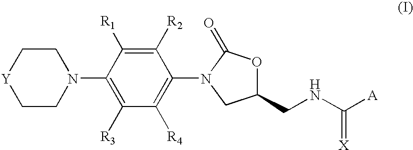
wherein:
—R₁, —R₂, —R₃ and —R₄ are radicals independently selected from hydrogen, F and Cl;
-A is a radical selected from the group consisting of 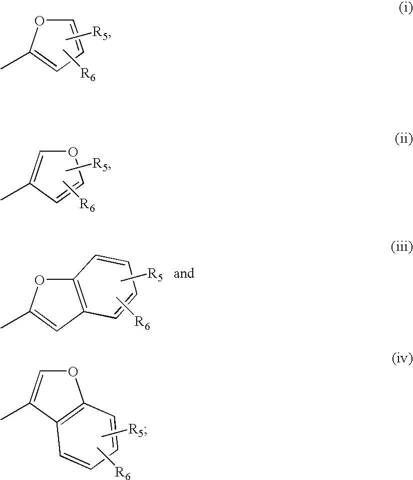
—R₅ and —R₆ are radicals independently selected from the group consisting of hydrogen, F, Cl, Br, —NO₂, —CN, —COR₇, —CSR₇, —SO₂R₇, —OCOR₇, alkyl(C₁-C₆), haloalkyl(C₁-C₆), cycloalkyl(C₃-C₆), alkenyl(C₂-C₆), alkynyl(C₂-C₆), alkoxyl(C₁-C₆), alkoxyalkyl(C₁-C₆), —NH-alkyl(C₁-C₆), —N-dialkyl(C₁-C₆), phenyl and heteroaryl; or R₅ and R₆ taken together form taken together form an optionally substituted benzo-fused ring optionally substituted;
—R₇ is a radical selected from the group consisting of hydrogen, alkyl(C₁-C₆), cycloalkyl(C₃-C₆), alkenyl(C₂-C₆), alkynyl(C₂-C₆), alkoxyl(C₁-C₆), alkoxyalkyl(C₁-C₆), hydroxyalkyl(C₁-C₆), —NH-alkyl(C₁-C₆), —N-dialkyl(C₁-C₆), phenyl and heteroaryl;
X is selected from O, S, NR₈ and CR₈R₉;
—R₈ and —R₉ are radicals independently selected from the group consisting of hydrogen, —CN, —COR₁₀, —SO₂R₁₀, alkyl(C₁-C₆), haloalkyl(C₁-C₆), cycloalkyl(C₃-C₆), alkenyl(C₂-C₆), alkynyl(C₂-C₆), alkoxyl(C₁-C₆), alkoxyalkyl(C₁-C₆), —NH-alkyl(C₁-C₆), —N-dialkyl(C₁-C₆), phenyl and heteroaryl;
—R₁₀ is a radical selected from the group consisting of hydrogen, alkyl(C₁-C₆), haloalkyl(C₁-C₆), cycloalkyl(C₃-C₆), alkenyl(C₂-C₆), alkynyl(C₂-C₆), alkoxyalkyl(C₁-C₆), phenyl and heteroaryl;
—Y— is a biradical selected from O, S, SO, SO₂, NO, NR₁₁, and CR₁₁R₁₂;
—R₁₁, and —R₁₂ are radicals independently selected from the group consisting of hydrogen, —(CHR₁₃)ₙR₁₄, —CN, —COR₁₃, —CSR₁₃, —COOR₁₃, —CSOR₁₃, —CONR₁₃R₁₄, —CSNR₁₃R₁₄, —CON(R₁₅)N(R₁₄)R₁₃, —SO₂R₁₃, —SO₂OR₁₃, —SO₂NR₁₃R₁₄, alkyl(C₁-C₆), haloalkyl(C₁-C₆), cycloalkyl(C₃-C₆), alkenyl(C₂-C₆), alkynyl(C₂-C₆), alkoxyalkyl(C₁-C₆), phenyl and heteroaryl;
n is selected from 0 and 1;
—R₁₃ and —R₁₄ are radicals independently selected from the group consisting of hydrogen, —COR₁₅, —CSR₁₅, —SO₂R₁₅, alkyl(C₁-C₆), cycloalkyl(C₃-C₆), alkenyl(C₂-C₆), alkynyl(C₂-C₆), alkoxyl(C₁-C₆), alkoxyalkyl(C₁-C₆), hydroxyalkyl(C₁-C₆), phenyl, 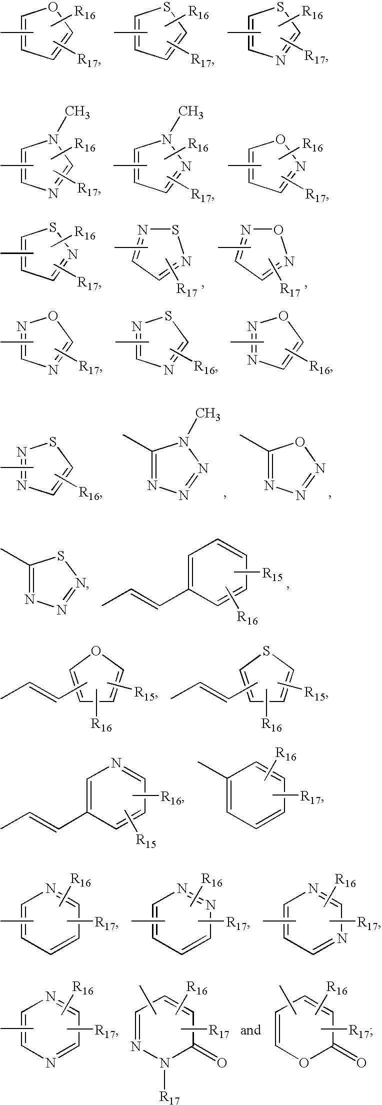
—R₁₅ is a radical selected from the group consisting of hydrogen, alkyl(C₁-C₆), cycloalkyl(C₃-C₆), alkenyl(C₂-C₆), alkynyl(C₂-C₆), alkoxyl(C₁-C₆), alkoxyalkyl(C₁-C₆), hydroxyalkyl(C₁-C₆), phenyl and heteroaryl;
—R₁₆ and —R₁₇ are radicals independently selected from the group consisting of F, Cl, Br, —NO₂, —CN, —COR₁₈, —CONR₁₈R₁₉, —SO₂R₁₈, —SO₂NR₁₈R₁₉, alkyl(C₁-C₆), haloalkyl(C₁-C₆), cycloalkyl(C₃-C₆), alkenyl(C₂-C₆), alkynyl(C₂-C₆), alkoxyl(C₁-C₆), alkoxyalkyl(C₁-C₆), phenyl and heteroaryl; and
—R₁₈ and —R₁₉ are radicals independently selected from the group consisting of hydrogen, alkyl(C₁-C₆), haloalkyl(C₁-C₆), cycloalkyl(C₃-C₆), alkenyl(C₂-C₆), alkynyl(C₂-C₆), alkoxyl(C₁-C₆), alkoxyalkyl(C₁-C₆), phenyl and heteroaryl.

3. A compound according to claim 1, wherein —R₂, —R₃ and —R₄ are hydrogen and —R₁ is F.

4. A compound according to claim 3, wherein X is O.

5. A compound according to claim 3, wherein X is S.

6. A compound according to claim 3, wherein X is N—CN.

7. A compound according to claim 3, wherein -A is the radical:

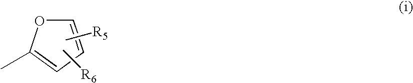

8. A compound according to claim 7, wherein —R₅ and —R₆ are hydrogen.

9. A compound according to claim 7, wherein —R₅ is hydrogen and —R₆ is selected from F, Cl and Br.

10. A compound according to claim 3, wherein -A is the radical:

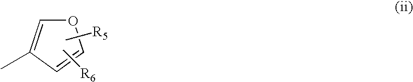

11. A compound according to claim 10, wherein —R₅ and —R₆ are hydrogen.

12. A compound according to claim 10, wherein —R₅ is hydrogen and —R₆ is selected from F, Cl and Br.

13. A compound according to claim 3, wherein -A is the radical:

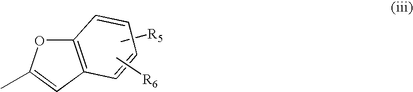

14. A compound according to claim 13, wherein —R₅ and —R₆ are hydrogen.

15. A compound according to claim 13, wherein —R₅ is hydrogen and —R₆ is NO₂.

16. A compound according to claim 3, wherein -A is the radical:

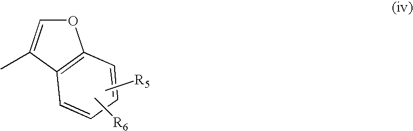

17. A compound according to claim 16, wherein —R₅ and —R₆ are hydrogen.

18. A compound according to claim 16, wherein —R₅ is hydrogen and —R₆ is selected from NO₂, F, Cl and Br.

19. A compound according to claim 3, wherein —Y— is a biradical selected from the group consisting of O, S, SO and SO₂.

20. A compound according to claim 3, wherein —Y— is NR₁₁.

21. A compound according to claim 20, wherein —R₁₁ is selected from the group consisting of hydrogen, methyl and ethyl.

22. A compound according to claim 20, wherein —R₁₁ is selected from the group consisting of —CN, —COCH₃, —COOCH₃, —CONHCH₃, —SO₂CH₃, and —SO₂NHCH₃.

23. A compound according to claim 20, wherein —R₁₁ is a radical selected from:

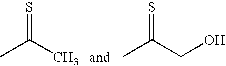

24. A compound according to claim 20, wherein —R₁₁ is a radical selected from

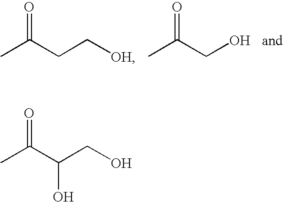

25. A compound according to claim 20, wherein —R₁₁, is a radical selected from:

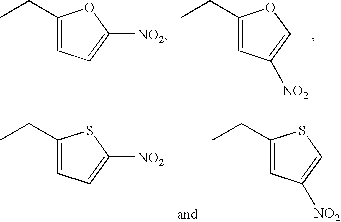

26. A compound according to claim 20, wherein —R₁₁ is a radical selected from:

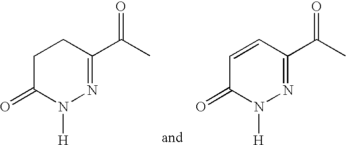

27. A compound according to claim 20, wherein —R₁₁ is the radical:

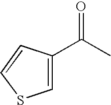

28. A compound according to claim 20, wherein —R₁₁ is the radical:

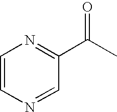

29. A compound according to claim 20, wherein —R₁₁ is the radical:

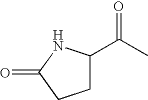

30. A compound according to claim 20, wherein —R₁₁ is a radical selected from:

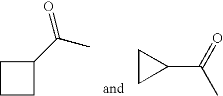

31. A compound according to claim 20, wherein —R₁₁ is a radical selected from:

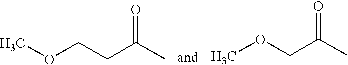

32. A compound according to claim 20, wherein —R₁₁, is a radical selected from:

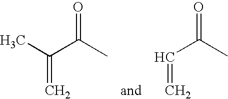

33. The compounds of claim 1 which are the enantiomers having the S-configuration at C-5 position of the oxazolidinone ring.

34. The compound according to claim 3, which is N-[[(5S)-3-[3-fluoro-4-(4′-thioacetyl-4-piperazinyl)-phenyl]-2-oxo-5-oxazolidinyl]methyl]furan-3-yl-thioamide of formula:

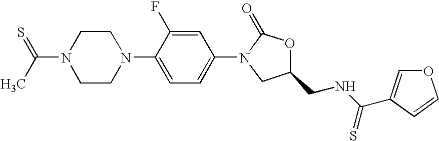

35. The compound according to claim 3, which is N-[[(5S)-3-[3-fluoro-4-morpholin-4-yl-phenyl]-2-oxo-5-oxazolidinyl]methyl]furan-3-yl-thioamide of formula:

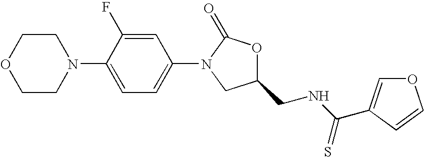

36. The compound according to claim 3, which is N-[[(5S)-3-[3-fluoro-4-(1-oxo-thiomorpholin-4-yl)-phenyl]-2-oxo-5-oxazolidinyl]methyl]furan-3-yl-thioamide of formula:

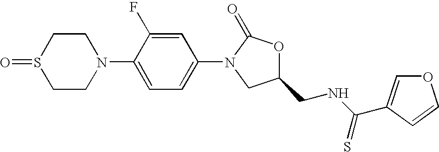

37. The compound according to claim 3, which is N-[[(5S)-3-[3-fluoro-4-morpholin-4-yl-phenyl]-2-oxo-5-oxazolidinyl]methyl]furan-2-yl-thioamide of formula:

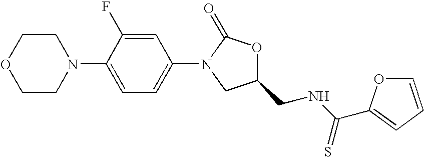

38. The compound according to claim 3, which is N-[[(5S)-3-[3-fluoro-4-(1-oxo-thiomorpholin-4-yl)-phenyl]-2-oxo-5-oxazolidinyl]methyl]furan-2-yl-thioamide of formula:

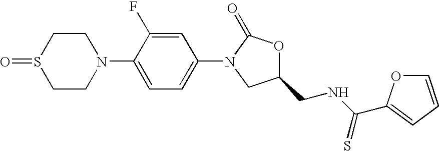

39. The compound according to claim 3, which is N-[[(5S)-3-[3-fluoro-4-morpholin-4-yl-phenyl]-2-oxo-5-oxazolidinyl]methyl]benzofuran-2-yl-amide of formula:

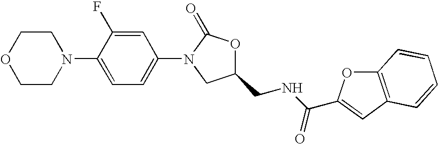

40. The compound according to claim 3, which is N-[[(5S)-3-[3-fluoro-4-morpholin-4-yl-phenyl]-2-oxo-5-oxazolidinyl]methyl]benzofuran-3-yl-amide of formula:

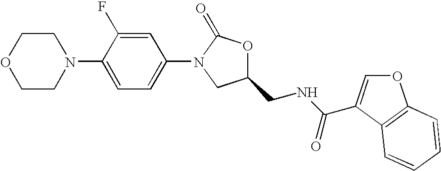

41. The compound according to claim 3, which is N-[[(5S)-3-[3-fluoro-4-morpholin-4-yl-phenyl]-2-oxo-5-oxazolidinyl]methyl]5-nitro-benzofuran-2-yl-amide of formula:

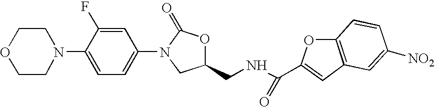

42. The compound according to claim 3, which is N-[[(5S)-3-[3-fluoro-4-(4′-methoxyacetyl-4-piperazinyl)-phenyl]-2-oxo-5-oxazolidinyl]methyl]furan-3-yl-amide of formula:

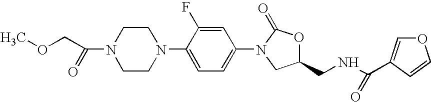

43. The compound according to claim 3, which is N-[[(5S)-3-[3-fluoro-4-(4′-acryloyl-4-piperazinyl)-phenyl]-2-oxo-5-oxazolidinyl]methyl]furan-3-yl-amide of formula:

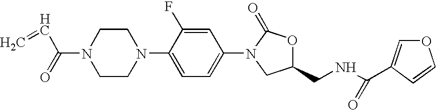

44. The compound according to claim 3, which is N-[[(5S)-3-[3-fluoro-4-(4′-hydroxyacetyl-4-piperazinyl)-phenyl]-2-oxo-5-oxazolidinyl]methyl]furan-3-yl-amide of formula:

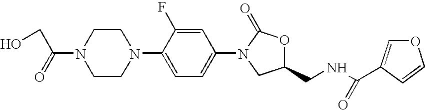

45. A method for the preparation of a compound of general formula (I), wherein —R₁, —R₂, —R₃, —R₄ and A have the meaning defined in claim 1, X is O, and —Y— is selected from O, S, SO and SO₂, which comprises acylating an amino methyl intermediate of general formula (II) 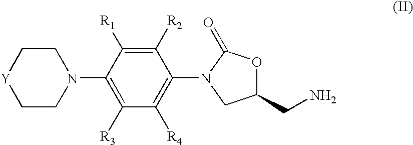
wherein —R₁, —R₂, —R₃, —R₄ and Y are as defined above, with an activated form of the corresponding acid of formula (III): 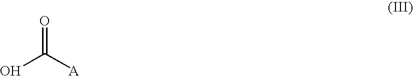
wherein -A is as defined in the general formula (I).

46. The method of claim 45 wherein the activated form of the acid (III) is selected from acid halides, imidazolides, p-nitrophenyl esters and 2,4,5-trichlorophenyl esters.

47. The method of claim 46 wherein the activated form of the acid (III) is prepared in situ in the presence of a reagent selected from triphenylphosphine, bromotrichloromethane, dicyclohexylcarbodiimide, 2-chloropyridinium cation, 3-chloroisoxazolium cation, diphenylphosphoryl azide, N-hydroxybenzotriazole (HOBt), 2-(1H-benzotriazole-1-yl)-1,1,3,3-tetramethyluronium hexafluorophosphate (HBTU), 1-(mesitylene-2-sulfonyl)-3-nitro-1H-1,2,4-triazole (MSNT), benzotriazole-1-yl-oxy-trispyrrolidino-phosphonium hexafluorophosphate (PyBOP), 1-ethyl-3-(3′-dimethylaminopropyl)carbodiimide HCl (WSC.HCl) and 2-(1H-benzotriazole-1-yl)-1,1,3,3-tetramethyluronium tetrafluoroborate (TBTU).

48. A method for the preparation of a compound of general formula (I), wherein —R₁, —R₂, —R₃, —R₄ and -A have the meaning defined in claim 1, X is O and —Y— is NH, which comprises:
(a) acylating an amino methyl intermediate of general formula (IIa) 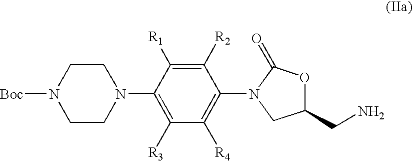
wherein —R₁, —R₂, —R₃ and —R₄ are as defined above and Boc is a t-butoxycarbonyl N-protecting group, with the corresponding acid of formula (III) wherein -A is as defined above, 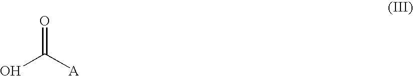
in the presence of 3-dimethylaminopropyl-3-ethyl-carbodiimide hydrochloride and 4-(dimethylamino)pyridine, thus obtaining the intermediate compound of formula (Ia) 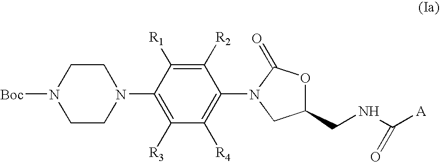
wherein -A, Boc, —R₁, —R₂, —R₃, and —R₄ are as defined above; and
(b) splitting off the Boc N-protecting group in (Ia) with trifluoroacetic acid.

49. A method for the preparation of a compound of general formula (I), wherein —R₁, —R₂, —R₃, —R₄ and -A have the meaning defined in claim 1, X is O, —Y— is NCOR₁₃, and R₁₃ is as defined in claim 1, which comprises reacting a compound of general formula (I), wherein X is O and Y is NH, with an activated form of the corresponding acid of formula (VI) 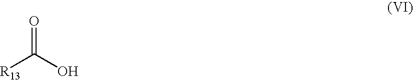
wherein —R₁₃ is as defined above.

50. The method of claim 49 wherein the activated form of the acid (VI) is selected from acid halides, imidazolides, p-nitrophenyl esters and 2,4,5-trichlorophenyl esters.

51. The method of claim 50 wherein the activated form of the acid (VI) is prepared in situ in the presence of a reagent selected from triphenylphosphine, bromotrichloromethane, dicyclohexylcarbodiimide, 2-chloropyridinium cation, 3-chloroisoxazolium cation, diphenylphosphoryl azide, N-hydroxybenzotriazole (HOBt), 2-(1H-benzotriazole-1-yl)-1,1,3,3-tetramethyluronium hexafluorophosphate (HBTU), 1-(mesitylene-2-sulfonyl)-3-nitro-1H-1,2,4-triazole (MSNT), benzotriazole-1-yl-oxy-trispyrrolidino-phosphonium hexafluorophosphate (PyBOP), 1-ethyl-3-(3′-dimethylaminopropyl)carbodiimide HCl (WSC.HCl) and 2-(1H-benzotriazole-1-yl)-1,1,3,3-tetramethyluronium tetrafluoroborate (TBTU).

52. A method for the preparation of a compound of general formula (I), wherein —R₁, —R₂, —R₃, —R₄, —Y— and -A have the meaning defined in claim 1, and X is S, which comprises reacting the corresponding compound of general formula (I), wherein X is O, with a thionation reagent selected from:

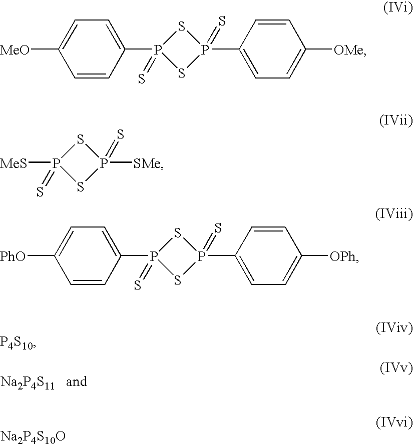

53. The method according to claim 52, wherein the thionation reagent is the Lawesson's reagent of formula (IVi):

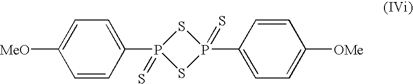

54. A method for the preparation of a compound of general formula (I), wherein —R₁, —R₂, —R₃, —R₄, —Y— and -A have the meaning defined in claim 1, and X is S, which comprises reacting the corresponding amino methyl derivative (II): 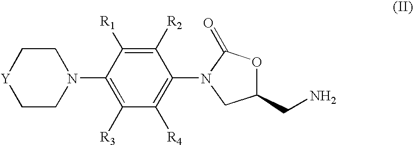
wherein —R₁, —R₂, —R₃, —R₄ and —Y— are as defined above, with an alkyldithioamide (IIIi): 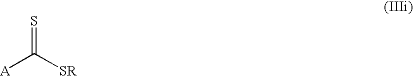
wherein -A is as defined above and —R is an alkyl(C₁-C₆).

55. A method for the preparation of a compound of general formula (I), wherein —R₁, —R₂, —R₃, —R₄, —Y— and -A have the meaning defined in claim 1, and Y is SO, which comprises oxidizing the corresponding compound of general formula (I) wherein —Y— is S, with a reagent selected from sodium metaperiodate, hypervalent iodine reagents, chromic acid in acetic acid or pyridine, lead tetraacetate, manganese dioxide, thallium (III) nitrate and ozone.

56. The method according to claim 55, wherein the reagent is sodium metaperiodate.

57. A method for the preparation of a compound of general formula (I), wherein —R₁, —R₂, —R₃, —R₄, —Y— and -A have the meaning defined in claim 1, and —Y— is SO₂, according to claim 1, which comprises oxidizing the corresponding compound of general formula (I), wherein —Y— is S, with a reagent selected from an excess of hydrogen peroxide in acetic acid and catalytic osmium tetroxide in the presence of N-methylmorpholine N-oxide.

58. The method according to claim 57, wherein the reagent is an excess of hydrogen peroxide in acetic acid.

59. A method for the preparation of a compound of general formula (I), wherein —R₁, —R₂, —R₃, —R₄, —Y— and -A have the meaning defined in claim 1, and X is N—CN, which comprises reacting an amino methyl intermediate of general formula (II): 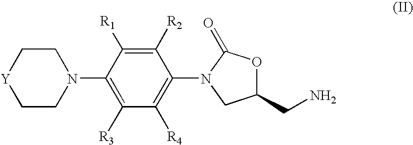
wherein —R₁, —R₂, —R₃, —R₄ and —Y— are as defined above, with a cyanoimidate of general formula (V): 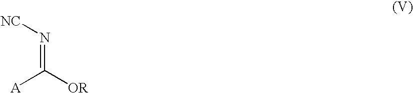
wherein -A is as defined above and —R is an alkyl(C₁-C₆).

60. Use of a compound of claim 1, for the preparation of a pharmaceutical composition to treat bacterial infections in a human or animal body.

61. Use according to claim 60, wherein the pharmaceutical composition is administered by the oral, parenteral, inhalatory, rectal, transdermal or topical route.

62. Use according to claim 60, wherein the compound is administered in an amount of 0.1 to 100 mg/kg of body weight/day.

63. Use according to claim 62, wherein the compound is administered in an amount of 1 to 50 mg/kg of body weight/day.

64. A pharmaceutical composition comprising a therapeutically effective amount of the compound of general formula (I) as defined in claim 1, together with the appropriate amounts of pharmaceutical excipients or carriers.
